(19) United States
(12) Patent Application Publication
(10) Pub. No.: US 2025/0302743 A1
(43) Pub. Date: Oct. 2, 2025

(54) METHODS OF TREATING DERMATOLOGICAL CONDITIONS AND SYMPTOMS THEREOF

(71) Applicant: KAMARI PHARMA LTD., Ness Ziona (IL)

(72) Inventors: Liora BRAIMAN-WIKSMAN, Ness Ziona (IL); Ephraim BRENER, Ness Ziona (IL); Boris VAISMAN, Ness Ziona (IL)

(21) Appl. No.: 18/861,802

(22) PCT Filed: May 5, 2023
(86) PCT No.: PCT/IB2023/054708

§ 371 Date: Oct. 30, 2024

Related U.S. Application Data
(60) Provisional application No. 63/338,586, filed on May 5, 2022.

(51) Int. Cl. A61K 9/06; A61K 9/00; A61K 9/107; A61K 31/4439; A61K 47/10; A61K 47/12; A61K 47/14; A61K 47/26; A61K 47/32; A61P 17/04

(52) U.S. Cl. CPC A61K 9/06; A61K 9/0014; A61K 9/107; A61K 31/4439; A61K 47/10; A61K 47/12; A61K 47/14; A61K 47/26; A61K 47/32; A61P 17/04

(57) ABSTRACT

Topically applied compositions comprising TRPV3 Inhibitors such as KM-001 significantly reduce the severity of dermatological lesions, pruritus, and combinations thereof in patients suffering from dermatological disorders such as keratodermas, lichen simplex chronicus, and ichthyoses.

### CROSS REFERENCE TO RELATED APPLICATION

The present application claims priority to and the benefit of U.S. Provisional Application No. 63/338,586, filed May 5, 2022, the disclosure of which is herein incorporated by reference in its entirety for all purposes.

### BACKGROUND

For skin to function properly, an intricate homeostasis of epidermal keratinocyte layers must be preserved, including proliferation, differentiation and cell signaling (Proksch E, Brandner J M, and Jensen J M. The skin: an indispensable barrier, Exp Dermatol, 2008; 17:1063-72). Damage to this homeostasis leads to dermatological diseases, which often present themselves as life-threatening conditions. One of the major factors dictating skin health is proper calcium homeostasis, largely regulated by membrane calcium ions channels (Elsholz F, Harteneck C, Muller W, and Friedland K. Calcium—a central regulator of keratinocyte differentiation in health and disease. Eur J Dermatol. 2014; 24: 650-61). Transient receptor potential cation channel, subfamily V, member 3 (TRPV3) is a cation channel displaying relatively high permeability to calcium. It was first cloned in 2002 and identified as a novel drug target in skin providing a potential new approach for the treatment of keratodermas and itch (Bakthavatchalam, Rajagopal, and S. David Kimball. 2010. Chapter 3—Modulators of Transient Receptor Potential Ion Channels. in John E. Macor (ed.), Annual Reports in Medicinal Chemistry (Academic Press); Broad L M, Mogg A J, Eberle E, Tolley M, Li D L, Knopp K L. TRPV3 in Drug Development. Pharmaceuticals (Basel). 2016 Sep. 9; 9(3):55). TRPV3 has been shown to be a central regulator in skin disease as it regulates proliferation, differentiation, and apoptosis of human epidermal keratinocytes. Specifically, its activation results in inhibition of keratinocyte proliferation and induction of apoptosis (Borbiró I, Lisztes E, Tóth BI, Czifra G, Oláh A, Szöllosi AG, Szentandrássy N, Nánási PP, Peter Z, Paus R, Kovács L, Biró T. Activation of transient receptor potential vanilloid-3 inhibits human hair growth. J Invest Dermatol. 2011 August; 131(8):1605-14). It has been implicated in the control of keratinocyte migration and wound healing, most probably via the release of nitric oxide (NO) (Miyamoto T, Petrus M J, Dubin A E, and Patapoutian A. TRPV3 regulates nitric oxide synthase-independent nitric oxide synthesis in the skin, Nat Commun 2011; 2: 369). It is also involved in both hair morphogenesis and hair follicle cycling and plays a role in maintenance of the skin barrier, as deletion of TRPV3 evokes deleterious changes in epidermal barrier structure (Moqrich A, Hwang S W, Earley T J, Petrus M J, Murray A N, Spencer K S, Andahazy M, Story G M, Patapoutian A. Impaired thermosensation in mice lacking TRPV3, a heat and camphor sensor in the skin. Science. 2005 Mar. 4; 307(5714):1468-72).

Various studies have provided several insights into how TRPV3 regulates keratinocyte structure and function. A key protein interaction partner appears to be the EGFR (Cheng X, Jin J, Hu L, Shen D, Dong X P, Samie M A, et al. TRP channel regulates EGFR signaling in hair morphogenesis and skin barrier formation. Cell. 2010; 141: 331-43). This receptor is proposed to form a signaling complex with TRPV3, whereby activation of the EGFR results in increased TRPV3 channel activity, stimulation of downstream secondary messengers and epidermal homeostasis. In the case of keratodermas such as punctate palmoplantar keratoderma (PPPK), the identified AAGAB mutation and its link to EGFR activation may lead to constant sensitization of TRPV3 and to uninterrupted influx of Ca⁺² ions into the cell. Similarly, Keratin 16 (Krt16), mutated in PC, is a direct target for EGFR and Erk1/2-mediated signaling, and its overexpression in mice dose-dependently enhances EGFR activity (Wang Y N, and Chang W C. Induction of disease-associated keratin 16 gene expression by epidermal growth factor is regulated through cooperation of transcription factors Sp1 and c-Jun. J Biol Chem. 2003; 278:45848-57; Chen Y J, Wang Y N, and Chang W C. ERK2-mediated C-terminal serine phosphorylation of p300 is vital to the regulation of epidermal growth factor-induced keratin 16 gene expression. J Biol Chem. 2007; 282: 27215-28). The proteolytically inactive rhomboid protein, iRhom2, is important in homeostasis of palmoplantar epidermis and is a key regulator of Krt16 expression (Maruthappu T, Chikh A, Fell B, Delaney P J, Brooke M A, Levet C, et al. Rhomboid family member 2 regulates cytoskeletal stress-associated Keratin 16. Nat Commun. 2017; 8: 14174) and regulates cytoskeletal stress response, barrier integrity, and signaling by p63 and ADAM17/EGFR.

TRPV3 activity was also associated with certain pathological cutaneous conditions. Multiple ‘gain-of-function’ mutations of TRPV3, in which the TRPV3 channel remains constantly open, were identified in Olmsted syndrome (OS) (Yadav M, Goswami C. TRPV3 mutants causing Olmsted Syndrome induce impaired cell adhesion and nonfunctional lysosomes. Channels (Austin). 2017 May 4; 11(3):196-208). This rare genodermatosis is characterized by the development of severe and sometimes mutilating palmoplantar keratoderma, periorificial hyperkeratotic plaques, diffuse alopecia, extreme pruritus, and pain (Duchatelet S, Hovnanian A. Olmsted syndrome: clinical, molecular and therapeutic aspects. Orphanet J Rare Dis. 2015; 17; 10:33). A murine strain DS-Nh with ‘gain-of-function’ TRPV3 mutation has been produced (Yoshioka T, Hikita I, Asakawa M, Hirasawa T, Deguchi M, Matsutani T, Oku H, Horikawa T, Arimura A. Spontaneous scratching behaviour in DS-Nh mice as a possible model for pruritus in atopic dermatitis. Immunology. 2006 July; 118(3):293-301). The DS-Nh mice spontaneously develop pruritus, which is associated with a dermatitis similar to human atopic dermatitis and OS.

Based on the mechanisms of action described above, inhibitors of TRPV3 activity possess an unexploited therapeutic potential for dermatological conditions such as keratodermas, dermatitis, and pruritus associated with or caused by dermatological conditions. TRPV3 inhibitors may improve keratinocyte proliferation and differentiation thus reducing hyperkeratosis and lesions.

Accordingly, developing safe and effective TRPV3 inhibitor compositions for treating dermatological conditions, and effective methods of administering such compositions to safely treat dermatological conditions would be of great benefit to patients.

### SUMMARY OF THE DISCLOSURE

The present disclosure provides a TRPV3 inhibitor composition and a method of treating a dermatological disorder.

In embodiments, the present disclosure provides a composition, comprising:

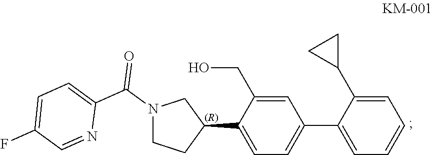

an aqueous phase; and an oil phase, wherein the concentration of KM-001 is about 0.1 wt. % to about 5.6 wt. %. In embodiments, the composition is a cream. In embodiments, the concentration of KM-001 is about 0.2 wt. % to about 3 wt. %. In embodiments, the concentration of KM-001 is about 0.3 wt. %. In embodiments, the concentration of KM-001 is about 1 wt. %.

In embodiments, the pH of the cream is about 4 to about 6. In embodiments, the pH of the cream is about 4.5 to about 5.5.

In embodiments, the cream comprises: (a) about 20 wt. % to about 40 wt. % of the oil phase; and (b) about 60 wt. % to about 80 wt. % of the aqueous phase. In embodiments, the oil phase comprises triglyceride, C₁₃₋₂₁ fatty alcohol, or a mixture thereof. In embodiments, the concentration of triglyceride is about 9 wt. % to about 13 wt. % of triglyceride. In embodiments, the concentration of triglyceride is about 11 wt. %. In embodiments, the triglyceride is medium chain triglyceride (MCT). In embodiments, the MCT is caprylic capric triglyceride. In embodiments, the concentration of C₁₃₋₂₁ fatty alcohol is about 10 wt. % to about 14 wt. %. In embodiments, the concentration of C₁₃₋₂₁ fatty alcohol is about 12 wt. %. In embodiments, the C₁₃₋₂₁ fatty alcohol is octyldodecanol. In embodiments, the oil phase is free of polyoxypropylene stearyl ether.

In embodiments, the cream further comprises an antioxidant, a preservative, a viscosity modifying agent, a pH modifier, or a mixture thereof. In embodiments, the antioxidant is propyl gallate, butylated hydroxyanisole (BHA), butylated hydroxytoluene, or a mixture thereof. In embodiments, the antioxidant comprises propyl gallate and butylated hydroxyanisole (BHA). In embodiments, the concentration of propyl gallate is about 0.02 wt. % to about 0.08 wt. % and the concentration of BHA is about 0.05 wt. % to about 0.2 wt. %. In embodiments, the cream comprises about 0.05 wt. % of propyl gallate and 0.1 wt. % of BHA.

In embodiments, the concentration of preservative is about 0.1 wt. % to about 2 wt. %. In embodiments, the preservative is a glycol ether, a phenol ether, a salt of benzoic acid, or a mixture thereof. In embodiments, the concentration of preservative is about 0.05 wt. % to about 0.4 wt. % phenoxyethanol, about 0.5 wt. % to about 2 wt. % sodium benzoate, or a mixture thereof. In embodiments, the viscosity modifying agent is a copolymer of acrylamide and sodium acryloyldimethyltaurate, polyoxyethylene sorbitan monooleate, sorbitan oleate, or a mixture thereof. In embodiments, the concentration of the viscosity modifying agent is about 1.5 wt. % to about 5 wt. % of the copolymer of acrylamide and sodium acryloyldimethyltaurate dispersed in isohexadecane. In embodiments, the pH modifier is 10% citric acid. In embodiments, the cream further comprises glycerin, propylene glycol, or a mixture thereof.

In embodiments, the viscosity of the cream is at least about 15,000 mPa·s, measured at 25° C. using a rotational viscosity method. In embodiments, the viscosity of the cream is about 15,000 mPa·s to about 75,000 mPa·s, measured at 25° C. using a rotational viscosity method. In embodiments, the viscosity of the cream is about 19,500 mPa·s to about 45,200 mPa·s, measured at 25° C. using a rotational viscosity method. In embodiments, the cream is an oil-in-water emulsion. In embodiments, the mean diameter of globules of the emulsion is less than about 13 μm. In embodiments, the mean diameter of globules of the emulsion is less than about 10 μm. In embodiments, the aqueous phase is a gel at about 2° C. to about 40° C.

In embodiments, topical administration of about 2 grams to about 4 grams of the cream comprising 1 wt. % of KM-001 to about 400 cm² to about 600 cm² of the skin of a patient in need thereof provides about 50 μg/g to about 6000 μg/g of the KM-001 in one or more skin layers, as measured by (matrix-assisted laser desorption/ionization) MALDI. In embodiments, topical administration of about 3 grams of the cream comprising 1 wt. % of KM-001 to about 500 cm² of the skin of a patient in need thereof provides about 50 μg/g to about 6000 μg/g of the KM-001 in one or more skin layers, as measured by (matrix-assisted laser desorption/ionization) MALDI.

In embodiments, topical administration of about 2 grams to about 4 grams of the cream comprising 1 wt. % of KM-001 to about 400 cm² to about 600 cm² of the skin of a patient in need thereof provides about 2000 μg/g to about 6000 μg/g of the KM-001 in epidermis, as measured by (matrix-assisted laser desorption/ionization) MALDI. In embodiments, topical administration of about 2 grams to about 4 grams of the cream comprising 1 wt. % of KM-001 to about 400 cm² to about 600 cm² of the skin of a patient in need thereof provides about 2000 g/g to about 6000 μg/g of the KM-001 in stratum basale, stratum spinosum, stratum granulosum, stratum lucidum, stratum corneum, or a combination thereof, as measured by (matrix-assisted laser desorption/ionization) MALDI. In embodiments, topical administration of about 2 grams to about 4 grams of the cream comprising 1 wt. % of KM-001 to about 400 cm² to about 600 cm² of the skin of a patient in need thereof provides about 500 μg/g to about 800 μg/g of the KM-001 in dermis, as measured by (matrix-assisted laser desorption/ionization) MALDI. In embodiments, topical administration of about 2 grams to about 4 grams of the cream comprising 1 wt. % of KM-001 to about 400 cm² to about 600 cm² of the skin of a patient in need thereof provides KM-001 in dermis at a concentration equivalent to about 1% to about 16% of the KM-001 in epidermis, as measured by MALDI. In embodiments, topical administration of about 2 grams to about 4 grams of the cream comprising 1 wt. % of KM-001 to about 400 cm² to about 600 cm² of the skin of a patient in need thereof provides about 100 g/g to about 3500 μg/g of the KM-001 in hypodermis, as measured by (matrix-assisted laser desorption/ionization) MALDI. In embodiments, topical administration of about 2 grams to about 4 grams of the cream comprising 1 wt. % of KM-001 to about 400 cm² to about 600 cm² of the skin of a patient in need thereof provides about 50 μg/g to about 1200 μg/g of the KM-001 in hair follicles, as measured by (matrix-assisted laser desorption/ionization) MALDI.

In embodiments, topical administration of the cream to the skin of a patient in need thereof provides penetration of about 0.05 wt. % to about 20 wt. % of the KM-001 presented in the applied composition in the skin into stratum corneum, epidermis, or dermis, as measured by in vitro penetration test (IVPT). In embodiments, the topical administration to the skin of a patient in need thereof provides penetration of about 1 wt. % to about 10 wt. % of the KM-001 presented in the applied composition in the skin into the stratum corneum, as measured by IVPT. In embodiments, the topical administration to the skin of a patient in need thereof provides penetration of about 0.2 wt. % to about 8 wt. % of the KM-001 presented in the applied composition in the skin into the epidermis, as measured by IVPT. In embodiments, the topical administration to the skin of a patient in need thereof provides penetration of about 0.05 wt. % to about 3 wt. % of the KM-001 presented in the applied composition in the skin into the dermis, as measured by IVPT.

In embodiments, the topical administration provides less than about 40 ng/mL of the maximum plasma concentration (Cₘₐₓ) of KM-001. In embodiments, the topical administration provides less than about 500 ng/mL*h of the area under the plasma drug concentration-tine curve (AUC) of KM-001.

In embodiments, the present disclosure provides method of treating a skin disorder or pruritus, comprising topically administering to the skin of a patient in need thereof a therapeutically effective amount of a KM-001-containing composition described herein in.

### BRIEF DESCRIPTION OF DRAWINGS

FIG. 1 shows the KM-001 distribution in placebo-treated minipig skin sample (MSI, overlay, and H&E images).

FIG. 2 shows the KM-001 distribution in KM-001 1% F3 treated minipig skin sample (MSI, overlay, and H&E images).

FIG. 3 shows the KM-001 distribution in KM-001 1% F4 treated minipig skin sample (MSI, overlay, and H&E images).

FIG. 4 shows the KM-001 distribution in KM-001 1% ointment treated minipig skin sample (MSI, overlay, and H&E images).

FIG. 5 shows the matrix-assisted laser desorption/ionization (MALDI) images of porcine skin tissue sections as described in Example 2.

FIG. 6 shows the comparative effect of KM-001 on scratching behavior in phenotypic DS-Nh mice.

FIG. 7 shows the comparative effect of KM-001 on skin pathology developed by DS-Nh mice.

FIG. 8 shows the effect of test treatments in skin morphology of DS-Nh mice.

FIG. 9A shows the visual appearance of the exemplary compositions M1, M2 and M3 as described in Example 9 and their respective vehicles (P—placebo) after incubation 24 h at 25° C.

FIG. 9B shows the visual appearance of exemplary compositions M6, M10, M13 and M14 and their respective vehicles (P—placebo) after incubation 24 h at 25° C.

FIG. 10 shows the definition of RapidOxy induction period.

FIG. 11A shows the mean blood levels of test items after dermal administration in male minipigs.

FIG. 11B shows the mean blood levels of test items after dermal administration in female minipigs.

FIGS. 12A-D shows the improvement of the affected area in pachyonychia congenita patients at timepoints: screening visit/baseline (T0) (FIG. 12A); visit 3 (FIG. 12B); visit 8 (FIG. 12C); and additional visit 8 (FIG. 12D).

### DETAILED DESCRIPTION

### Definitions

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

As used herein, the term “about” refers to plus or minus 10% of the referenced number unless otherwise stated or otherwise evident by the context, and except where such a range would exceed 100% of a possible value, or fall below 0% of a possible value, such as less than 0% content of an ingredient, or more than 100% of the total contents of a composition. For example, reference to an amount of any of the TRPV3 inhibitors disclosed herein of “about 1 wt. %” means that the TRPV3 inhibitor can be present at any amount ranging from 0.9% to 1.1% by weight of the composition. In embodiments, the terms “wt. %” and “% w/w” are used interchangeably.

The term “a” or “an” refers to one or more of that entity; for example, “a solvent” refers to one or more solvents or at least one solvent. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, the term “skin” refers to any of the layers of the skin, including the epidermis, dermis, and hypodermis. The epidermis has five sub-layers, including the stratum corneum, stratum lucidum, stratum granulosum, stratum spinosum, and stratum basale, which are listed from the outermost sub-layer to the innermost sub-layer. For example, the stratum corneum is the surface layer of the skin.

As used herein, the term “topical composition” refers to any formulation that is designed to be applied to the skin.

The term “aqueous” as used herein is a composition wherein the composition contains greater than 50% by weight of water. In embodiments, the aqueous compositions of the present disclosure contain greater than 75% by weight of water. In embodiments, the aqueous compositions of the present disclosure contain greater than 90% by weight of water.

The terms “effective amount” and “therapeutically effective amount” are used interchangeably in this disclosure and refer to an amount of a compound that, when administered to a patient, is capable of performing the intended result. For example, an effective amount of TRPV3 inhibitor in a composition is that amount that is required to reduce at least one symptom of a skin disorder in a patient. The actual amount that comprises the “effective amount” or “therapeutically effective amount” will vary depending on a number of conditions including, but not limited to, the severity of the disorder and the size and health of the patient, or the size of a wound to be treated. A skilled medical practitioner can readily determine the appropriate amount using methods known in the medical field.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “treat” or “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition), reducing the risk of developing or worsening a condition, or restoring or normalizing skin.

### Km-001 Compositions

In embodiments, the present disclosure is directed to compositions comprising any of the TRPV3 inhibitors disclosed in U.S. Patent Application Publication No. US 2002/0332697 A1, hereby incorporated by reference in its entirety. In US 2002/0332697 A1, one of the TRPV3 inhibitors, KM-001, was found to inhibit TRPV3 with an IC50 of ˜3 nM. The KM-001 compound also significantly reduced Ca⁺² flux in keratinocytes and normalized differentiation markers. However, the TRPV3 inhibitors disclosed in US 2002/0332697 A1 were not readily dissolved in aqueous compositions, resulting low bioavailability to the target cells and/or tissues. The present application, in embodiments, addresses this problem in the art and provides TRPV3 inhibitor compositions that are safe, bioavailable, and efficacious in treating dermatological disorders.

In embodiments, the compositions of the present disclosure comprise a TRPV3 inhibitor having the chemical structure of KM-001:

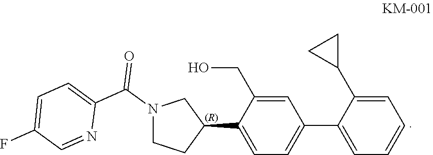

The compositions of the present disclosure are generally formulated for topical use, for example, as creams, ointments, pastes, lotions, or gels. Creams or lotions include emulsions of an aqueous phase and a hydrophobic (oily) phase and can be classified as oil-in-water (o/w) or water-in-oil (w/o) emulsions. In embodiments, the composition of the present disclosure is an oil-in-water emulsion. In o/w emulsions, the oil phase is discontinuous and dispersed in a continuous water phase, whereas in w/o emulsions, the water phase is discontinuous and dispersed in a continuous oil phase. Emulsifiers can be added to o/w or w/o emulsions to stabilize the emulsion and inhibit/slow phase separation that would destabilize the emulsion.

In embodiments, the compositions of the present disclosure are formulated as creams. In embodiments, the compositions of the present disclosure can be formulated as ointments, which are semi-solid preparations of hydrocarbons (e.g., petrolatum, mineral oil, paraffins, silicone oils and synthetic hydrocarbons), or pastes, which are mixtures of a powder and an ointment to improve porosity (breathability) and to make the paste more difficult to remove or migrate to areas of the skin that do not require treatment. In embodiments, gel compositions typically comprise viscous cellulose ethers or carbomers in a water-alcohol mixture. In embodiments, gels typically dry on the skin and leave a thin film containing the active ingredient (e.g., TRPV3 inhibitor), and can be more suitable for hairy areas of the skin compared to other types of topical formulations.

In embodiments, the compositions of the present disclosure comprise therapeutically effective amounts of any of the TRPV3 inhibitors of disclosed herein (e.g., KM-001 or KM-023). The concentration of the TRPV3 inhibitors of the present disclosure (e.g., KM-001 or KM-023) typically ranges from about 0.1 wt. % to about 6 wt. %, including about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.2 wt. %, about 1.4 wt. %, about 1.6 wt. %, about 1.8 wt. %, about 2.0 wt. %, about 2.2 wt. %, about 2.4 wt. %, about 2.6 wt. %, about 2.8 wt. %, about 3.0 wt. %, about 3.2 wt. %, about 3.4 wt. %, about 3.6 wt. %, about 3.8 wt. %, about 4.0 wt. %, about 4.2 wt. %, about 4.4 wt. %, about 4.6 wt. %, about 4.8 wt. %, about 5.0 wt. %, about 5.2 wt. %, about 5.4 wt. %, about 5.6 wt. %, about 5.8 wt. %, or about 6.0 wt. %, inclusive of all ranges between any of these values or ranges.

In embodiments, the compositions of the present disclosure are formulated as creams, gels, or gel-like creams. In embodiments, the present disclosure provides a cream, comprising:

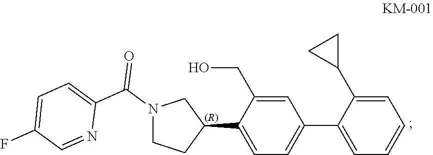

an aqueous phase; and an oil phase, wherein the concentration of KM-001 is about 0.1 wt. % to about 5.6 wt. %, for example, 0.2 wt. % to about 2 wt. %, about 0.3 wt. % to about 1.0 wt. %, about 0.5 wt. %, to about 5 wt. %, including about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, or about 5.6 wt. %, including all ranges between any of these values. In embodiments, the concentration of KM-001 is about 0.2 wt. % to about 3 wt. %. In embodiments, the concentration of KM-001 is about 0.3 wt. %. In embodiments, the concentration of KM-001 is about 1 wt. %.

In embodiments, the compositions (e.g., creams) of the present disclosure have a pH of about 4.0 to about 6.0, e.g., about 4.0, about 4.1, about 4.2, about 4.3, about 4.4, or about 4.5, about 4.6, about 4.7, about 4.8, about 4.9, about 5.0, about 5.1, about 5.2, about 5.3, about 5.4, or about 5.5, about 5.6, about 5.7, about 5.8, about 5.9, about 6.0, including any values or ranges therebetween. In embodiments, the compositions of the present disclosure have a pH of about 4.0 to about 5.0. In embodiments, the compositions of the present disclosure have a pH of about 4.5 to about 5.5. In embodiments, the compositions of the present disclosure have a pH of about 4.9 to about 5.1. In embodiments, the compositions of the present disclosure have a pH of about 4.0.

In embodiments, the cream comprises: (a) about 20 wt. % to about 40 wt. % of the oil phase; and (b) about 60 wt. % to about 80 wt. % of the aqueous phase. In embodiments, the composition (e.g., cream) comprises the oil phase at a wt. % of about 20 wt. % to about 40 wt. %, e.g., about 20.0 wt. %, about 20.2 wt. %, about 20.4 wt. %, about 20.6 wt. %, about 20.8 wt. %, about 30.2 wt. %, about 30.4 wt. %, about 30.6 wt. %, about 30.8 wt. %, or about 40.0 wt. %, including any values or ranges therebetween. In embodiments, the compositions comprise about 60 wt. % to about 80 wt. % of the aqueous phase, e.g., about 60.0 wt. %, about 60.2 wt. %, about 60.4 wt. %, about 60.6 wt. %, about 60.8 wt. %, about 70.0 wt. %, about 70.2 wt. %, about 70.4 wt. %, about 70.6 wt. %, about 70.8 wt. %, or about 80.0 wt. %, including any values or ranges therebetween.

In embodiments, composition is an oil-in-water emulsion. In embodiments, the aqueous phase is a gel at about 2° C. to about 40° C., for example, about 2° C., about 4° C., about 6° C., about 8° C., about 10° C., about 12° C., about 14° C., about 16° C., about 18° C., about 20° C., about 22° C., about 24° C., about 26° C., about 28° C., about 30° C., about 32° C., about 34° C., about 36° C., about 38° C., or about 40° C., including any values or ranges therebetween. In embodiments, the mean diameter of globules of the emulsion is less than about 13 μm, for example, less than about 13 μm, about 12.9 μm, about 12.8 μm, about 12.7 μm, about 12.6 μm, about 12.5 μm, about 12.4 μm, about 12.3 μm, about 12.2 μm, about 12.1 μm, about 12.0 μm, about 11.9 μm, about 11.8 μm, about 11.7 μm, about 11.6 μm, about 11.5 μm, about 11.4 μm, about 11.3 μm, about 11.2 μm, about 11.1 μm, about 11.0 μm, about 10.9 μm, about 10.8 μm, about 10.7 μm, about 10.6 μm, about 10.5 μm, about 10.4 μm, about 10.3 μm, about 10.2 μm, about 10.1 μm, or less than about 10.0 μm, including all values and ranges therebetween. In embodiments, the mean diameter of globules of the emulsion is less than about 13 μm. In embodiments, the mean diameter of globules of the emulsion is less than about 10 μm.

In embodiments, the oil phase comprises triglyceride, C₁₃₋₂₁ fatty alcohol, or a mixture thereof. In embodiments, the oil phase comprises triglyceride. In embodiments, the concentration of triglyceride is about 9 wt. % to about 13 wt. %, e.g., about 9.0 wt. %, about 9.1 wt. %, about 9.2 wt. %, about 9.5 wt. %, about 9.6 wt. %, about 9.7 wt. %, about 9.8 wt. %, about 9.9 wt. %, about 10.0 wt. %, about 10.1 wt. %, about 10.2 wt. %, about 10.3 wt. %, about 10.4 wt. %, about 10.5 wt. %, about 10.6 wt. %, about 10.7 wt. %, about 10.8 wt. %, about 10.9 wt. %, about 11.0 wt. %, about 11.1 wt. %, about 11.2 wt. %, about 11.3 wt. %, about 11.4 wt. %, about 11.5 wt. %, about 11.6 wt. %, about 11.7 wt. %, about 11.8 wt. %, about 11.9 wt. %, about 12.0 wt. %, about 12.1 wt. %, about 12.2 wt. %, about 12.3 wt. %, about 12.4 wt. %, about 12.5 wt. %, about 12.6 wt. %, about 12.7 wt. %, about 12.8 wt. %, about 12.9 wt. %, or about 13.0 wt. %, including all values and ranges therebetween. In embodiments, the concentration of triglyceride is about 11 wt. %. In embodiments, the triglyceride is a C₆-C₁₂ (e.g., C₆, C₇, C₈, C₉, C₁₀, C₁₁, or C₁₂) medium chain triglyceride (MCT). In embodiments, the MCT is caprylic capric triglyceride.

In embodiments, the oil phase comprises C₁₃₋₂₁ fatty alcohol (e.g., C₁₃, C₁₄, C₁₅, C₁₆, C₁₇, C₁₈, C₁₉, C₂₀, or C₂₁). In embodiments, the concentration of C₁₃₋₂₁ fatty alcohol is about 10 wt. % to about 14 wt. %, for example, about 10.0 wt. %, about 10.1 wt. %, about 10.2 wt. %, about 10.3 wt. %, about 10.4 wt. %, about 10.5 wt. %, about 10.6 wt. %, about 10.7 wt. %, about 10.8 wt. %, about 10.9 wt. %, about 11.0 wt. %, about 11.1 wt. %, about 11.2 wt. %, about 11.3 wt. %, about 11.4 wt. %, about 11.5 wt. %, about 11.6 wt. %, about 11.7 wt. %, about 11.8 wt. %, about 11.9 wt. %, about 12.0 wt. %, about 12.1 wt. %, about 12.2 wt. %, about 12.3 wt. %, about 12.4 wt. %, about 12.5 wt. %, about 12.6 wt. %, about 12.7 wt. %, about 12.8 wt. %, about 12.9 wt. %, about 13.0 wt. % about 13.1 wt. %, about 13.2 wt. %, about 13.3 wt. %, about 13.4 wt. %, about 13.5 wt. %, about 13.6 wt. %, about 13.7 wt. %, about 13.8 wt. %, about 13.9 wt. %, or about 14.0 wt. % including all values and ranges therebetween. In embodiments, the concentration of C₁₃₋₂₁ fatty alcohol is about 12 wt. %. In embodiments, the C₁₃₋₂₁ fatty alcohol is octyldodecanol.

In embodiments, the compositions or formulations of the present disclosure further comprise pharmacologically acceptable preservatives, antioxidants, solvents, viscosity enhancers (i.e., viscosity modifying agents), pH modifier (i.e., pH adjusting agents), emollients, humectants, coloring agents, and fragrances. In embodiments, the composition of the present disclosure further comprises an antioxidant, a preservative, a viscosity modifying agent, a pH modifier, or a mixture thereof.

In embodiments, the compositions (e.g., creams) of the present disclosure comprise an antioxidant. In embodiments, the antioxidants comprise phenolic compounds such as butylated hydroxytoluene (BHT), butylated hydroxyanisole (BHA), tertiary butylhydroquinone, natural antioxidants such as tocopherol (Vitamin E), ascorbic acid (vitamin C), polyphenols (e.g., propyl gallate), flavonoids, retinol (Vitamin A), or a mixture thereof. In embodiments, the antioxidant is propyl gallate, butylated hydroxyanisole (BHA), butylated hydroxytoluene, or a mixture thereof. In embodiments, the antioxidant is a mixture of propyl gallate and butylated hydroxyanisole (BHA). In embodiments, the concentration of propyl gallate is about 0.02 wt. % to about 0.08 wt. %, for example, about 0.02 wt. %, about 0.03 wt. %, about 0.04 wt. %, about 0.05 wt. %, about 0.06 wt. %, about 0.07 wt. %, or about 0.08 wt. %, including all values and ranges therebetween. In embodiments, the concentration of BHA is about 0.05 wt. % to about 0.2 wt. %, for example, about 0.05 wt. %, about 0.06 wt. %, about 0.07 wt. %, about 0.08 wt. %, about 0.09 wt. %, about 0.1 wt. %, 0.11 wt. %, about 0.12 wt. %, about 0.13 wt. %, about 0.14 wt. %, about 0.15 wt. %, about 0.16 wt. %, 0.17 wt. %, about 0.18 wt. %, about 0.19 wt. %, or about 0.2 wt. %, including all values and ranges therebetween. In embodiments, the composition comprises about 0.05 wt. % of propyl gallate and about 0.1 wt. % of BHA.

In embodiments, the compositions (e.g., creams) of the present disclosure comprise a preservative. In embodiments, the preservatives (e.g., to inhibit or prevent the growth of microorganisms, fungi, etc. in the topical formulation) comprise triclosan, methylisothiazolinone, methylchloroisothiazolinone, chlorphenesin, chloroxylenol, iodopropynylbutylcarbamate, methyldibromoglutaronitrile, formaldehyde, benzylhemiformal, diazolidinyl urea, imidazolidinyl urea, 2-bromo-2-nitropropane-1,3-diol, DMDM hydantoin, MDM hydantoin, quaternium-15, sodium hydroxymethyl glycinate, phenoxyethanol, 2-butoxyethanol, 2-(2-butoxyethoxy)ethanol, 2-(2-ethoxy)ethanol, methylparaben, ethylparaben, propylparaben, butylparaben, isobutylparaben, benzoic acid (and salts thereof), sorbic acid (and salts thereof), salicylic acid (and salts thereof), alcohols, or a mixture thereof. In embodiments, the concentration of the preservative is about 0.05 wt. % to about 2 wt. %, for example, about 0.05 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2.0 wt. %, including any values or ranges therebetween. In embodiments, the preservative is a glycol ether, a phenol ether, a salt of benzoic acid, or a mixture thereof. In embodiments, the preservative is phenoxyethanol. In embodiments, the preservative is sodium benzoate. In embodiments, the concentration of phenoxyethanol is about 0.05 wt. % to about 0.4 wt. %, for example, about 0.05 wt. %, about 0.1 wt. %, about 0.2 wt. %, about 0.3 wt. %, or about 0.4 wt. %, including any values or ranges therebetween. In embodiments, the concentration of sodium benzoate is about 0.5 wt. % to about 2 wt. %, for example, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2.0 wt. %, including any values or ranges therebetween. In embodiments, the preservative is about 0.05 wt. % to about 0.4 wt. % of phenoxyethanol, or about 0.5 wt. % to about 2 wt. % of sodium benzoate, or a mixture thereof.

In embodiments, the compositions (e.g., creams) of the present disclosure have a pH of about 4.5 to about 5.5 and comprises a preservative. In embodiments, the preservative is sodium benzoate, which provides antimicrobial activities at acidic pH.

In embodiments, the compositions (e.g., creams) of the present disclosure comprise a pharmaceutically acceptable solvent. In embodiments, the compositions of the present disclosure comprise acetone, 2-methylpentane-2,4-diol, propylene glycol, caprylic capric triglyceride (available from Oleochemicals as MIGLYOL 812N), octyldodecanol, limonene, 1,3-butanediol, 1,3-dioxolane, 1,3-propanediol, 1,5-pentanediol, 1,6-hexanediol, 1-decene, 1-heptanol, 1-hexanol, N-butylacetate, ethyl acetate, methyl acetate, dimethylisosorbide, alpha-terpineol, benzyl alcohol, diethyl sebacate, diethylene glycol monoethyl ether, diisopropyl adipate, isosorbide dimethyl ether, dimethyl sulfoxide, ethyl acetate, isopropyl tetradecanoate, N-methyl-2-pyrrolidone, oleic acid, polyethylene glycol 400, polysorbate 20, polysorbate 80, propylene carbonate, propylene glycol diacetate, acetonitrile, chlorobenzene, cyclohexane, 1,4-dioxane, methanol, ethanol, 2-methoxyethanol, methylbutyl ketone, methylcyclohexane, N,N-dimethylacetamide, N,N-dimethylformamide, 1,4-dioxane, nitromethane, pyridine, sulfolane, toluene, orxylene, or a mixture thereof.

In some embodiments, the compositions (e.g., creams) of the present disclosure comprise a thickening agent. In embodiments, thickening agents (i.e., viscosity modifying agents), including thickeners or gelling agents, comprise substances which that can increase the viscosity of a composition. In embodiments, the viscosity modifying agents comprise substances that can increase the viscosity of a composition without substantially modifying the efficacy of the active ingredient within the composition. In embodiments, viscosity modifying agents can also increase the physical stability of the compositions of the present disclosure. In embodiments, the viscosity enhancers, viscosity modifying agents, or thickening agents comprise lipid thickeners such as cetyl alcohol, stearyl alcohol, carnauba wax, stearic acid; naturally derived thickeners such as hydroxyethyl cellulose, acacia gum, guar gum, locust bean gum, xanthan gum, gelatin, hyaluronic acid, acacia, agar, algin, alginic acid, ammonium alginate, amylopectin, calcium alginate, calcium carrageenan, carnitine, carrageenan, dextrin, gellan gum, guar hydroxypropyltrimonium chloride, hydroxypropyl chitosan, hydroxypropyl guar, karaya gum, kelp, natto gum, potassium alginate, potassium carrageenan, propylene glycol alginate, sclerotium gum, sodium carboxymethyl dextran, sodium carrageenan, tragacanth gum; mineral thickeners such as hectorite, hydrated silica, silica, bentonite, magnesium aluminum silicate; synthetic thickeners such as carbomer (water-swellable acrylic acid polymer sold under trade names ULTREZ 30 (Lubrizol), ULTREZ 21 (Lubrizol), TEGO Carbomer 140 G (Evonik), and SEPINEO P600 (Acrylamide/AMPS copolymer; Seppic).

In embodiments, the viscosity modifying agent is a copolymer of acrylamide and sodium acryloyldimethyltaurate (e.g., SEPINEO P600), polyoxyethylene sorbitan monooleate, sorbitan oleate, or a mixture thereof. In embodiments, the viscosity modifying agent is a copolymer of acrylamide and sodium acryloyldimethyltaurate dispersed in isohexadecane. In embodiments, the concentration of the copolymer of acrylamide and sodium acryloyldimethyltaurate is about 1.5 wt. % to about 5 wt. % (for example, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5.0 wt. %, including any values or ranges therebetween). In embodiments, the viscosity modifying agent is about 1.5 wt. % to about 5 wt. % of the copolymer of acrylamide and sodium acryloyldimethyltaurate dispersed in isohexadecane, polyoxyethylene sorbitan monooleate, sorbitan oleate, or a mixture thereof.

In embodiments, viscosity modifying agents, or thickening agents in the compositions of the present disclosure comprise carboxylic acid polymers, cross-linked polyacrylate polymers, polyacrylamide polymers, polysaccharides, gums, or a mixture thereof. In embodiments, the carboxylic acid polymers comprise cross-linked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; hereby incorporated by references by their entireties, and CTFA International Cosmetic Ingredient Dictionary, Fourth edition, 1991, pp. 12 and 80).

In embodiments, the compositions of the present disclosure comprise a pH modifier. In embodiments, the pH modifier (i.e., pH-adjusters or pH modifying agents) comprises any pharmaceutically acceptable buffering agents capable of buffering in an acceptable pH range (e.g., pH 4-5) such as citric acid buffers and acetate buffers. In embodiments, the pH modifier is 10% citric acid.

In embodiments, the compositions (e.g., creams) of the present disclosure further comprises emollients. In embodiments, the pharmaceutically acceptable emollients (or moisturizers) in the compositions of the present disclosure comprise vegetable oils, petrolatum, cetyl alcohol, cetearyl alcohol, cholesterol, cocoa butter, shea butter, isopropyl myristate, isopropyl palmitate, lanolin, liquid paraffin, polyethylene glycols, shea butter, silicone oils, stearic acid, stearyl alcohol, castor oil, etc. In embodiments, the pharmaceutically acceptable humectant in the compositions of the present disclosure comprise hyaluronic acid, glycerin, alpha hydroxy acids (e.g., glycolic acid, lactic acid, citric acid), propylene glycol, aloe vera gel, erythritol, xylitol, sorbitol, fructose, glucose, glycerin, glycerol polymers, glycol, 1,2,6-hexanetriol, honey, hydrogenated honey, hydrogenated starch hydrolysate, inositol, lactitol, maltitol, maltose, mannitol, PEG-15 butanediol, polyglyceryl sorbitol, salts of pyrrolidone carboxylic acid, potassium PCA, propylene glycol, sodium glucouronate, sodium PCA, sucrose, trehalose, urea, panthenol (vitamin B5), peptides, amino acids, and salicylic acid. In embodiments, the cream comprises glycerin, propylene glycol, or a mixture thereof.

In embodiments, the compositions (e.g., creams) of the present disclosure optionally include chelating agents. In embodiments, the pharmaceutically acceptable chelating agents in the compositions of the present disclosure comprise disodium ethylenediaminetetraacetic acid (EDTA), tetrasodium EDTA or a mixture thereof.

In embodiments, the compositions (e.g., creams) of the present disclosure optionally include UV absorption agents. In embodiments, the chemical UV absorption agents in the compositions of the present disclosure comprise para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methylcinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloyl trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis-diethylamino hydroxybenzoyl benzoate, bis benzoxazoylphenyl ethylhexylimino-triazine, drometrizole trisiloxane, methylene bis-benzotriazolyl tetramethylbutylphenol, and bis-ethylhexyloxyphenol methoxyphenyltriazine, 4-methylbenzylidene camphor, isopentyl-4-methoxy cinnamate, or a mixture thereof. In embodiments, the physical UV absorption agents comprise kaolin, talc, petrolatum and metal oxides (e.g., titanium dioxide and zinc oxide), or a mixture thereof.

In embodiments, the viscosity of the compositions of the present disclosure is at least about 15,000 mPa·s, for example, at least about 15,500 mPa·s, about 16,000 mPa·s, about 17,000 mPa·s, about 18,000 mPa·s, about 19,000 mPa·s, about 20,000 mPa·s, about 25,000 mPa·s, about 30,000 mPa·s, about 35,000 mPa·s, about 40,000 mPa·s, about 45,000 mPa·s, about 50,000 mPa·s, about 55,000 mPa·s, about 60,000 mPa·s, about 65,000 mPa·s, or about 70,000 mPa·s, measured at 25° C. using a rotational viscosity method (Brookfield RVDV II viscometer) described in USP <912>.

In embodiments, the viscosity of the cream is about 15,000 mPa·s to about 90,000 mPa·s, for example, 15,000 mPa·s, about 20,000 mPa·s, about 25,000 mPa·s, about 30,000 mPa·s, about 35,000 mPa·s, about 40,000 mPa·s, about 45,000 mPa·s, about 50,000 mPa·s, about 55,000 mPa·s, about 60,000 mPa·s, about 65,000 mPa·s, about 70,000 mPa·s, about 75,000 mPa·s, about 80,000 mPa·s, about 85,000 mPa·s, or about 90,000 mPa·s, including any values or ranges therebetween, measured at 25° C. using a rotational viscosity method. In embodiments, the viscosity of the cream is about 15,000 mPa·s to about 75,000 mPa·s, measured at 25° C. using a rotational viscosity method. In embodiments, the viscosity of the cream is about 19,500 mPa·s to about 45,200 mPa·s, measured at 25° C. using a rotational viscosity method. In embodiments, the viscosity measurement is performed under a constant shear rate (rotation per minute) after a pre-conditioning of the sample under the same velocity. In embodiments, the viscosity of compositions is measured using Brookfield viscometer RVDVII+, small sample adaptor (SSA) and mobile number 34 from SSA spindle set.

In embodiments, the compositions of the present disclosure are topically administered to deliver a pharmaceutically effective level of the TRPV3 inhibitor (e.g., KM-001) to epidermis. In embodiments, the compositions of the present disclosure are topically administered to deliver a pharmaceutically effective level of the TRPV3 inhibitor (e.g., KM-001) to one or more of the suprabasal layers of epidermis (e.g., cornified envelopes, granular and spinous layers, and the upper dermis). In embodiments, topical administration of the compositions of the present disclosure provide penetration of the TRPV3 inhibitor to all of the suprabasal layers of epidermis. In embodiments, topical administration of the compositions of the present disclosure provides a local concentration of the TRPV3 inhibitor (e.g., KM-001) of about 60 μg/g to about 160 mg/g, for example, about 60 μg/g, about 70 μg/g, about 80 μg/g, about 90 μg/g, about 100 μg/g, about 200 μg/g, about 300 μg/g, about 400 μg/g, about 500 μg/g, about 600 μg/g, about 700 μg/g, about 800 μg/g, about 900 μg/g, about 1000 μg/g, about 5 mg/g, about 10 mg/g, about 15 mg/g, about 20 mg/g, about 25 mg/g, about 30 mg/g, about 35 mg/g, about 40 mg/g, about 45 mg/g, about 50 mg/g, about 55 mg/g, about 60 mg/g, about 65 mg/g, about 70 mg/g, about 75 mg/g, about 80 mg/g, about 85 mg/g, about 90 mg/g, about 95 mg/g, about 100 mg/g, about 110 mg/g, about 115 mg/g, about 120 mg/g, about 125 mg/g, about 130 mg/g, about 135 mg/g, about 140 mg/g, about 145 mg/g, about 150 mg/g, about 155 mg/g, or about 160 mg/g, including all ranges between any of these values.

After topical administration of the compositions of the present disclosure, the local concentration of the TRPV3 inhibitor (e.g., KM-001) in the dermis ranges from about 1.5 wt. % to 16 wt. % of the total amount of KM-001 in the skin as measured by IVPT method described herein, including about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, or about 16.0 wt. %, including all ranges between any of these values, of the total amount of KM-001 presented in the applied composition in the skin as measured using the IVPT method described herein.

In embodiments, the topical administration to the skin of a patient in need thereof provides penetration of about 0.05 wt. % to about 20 wt. %, for example, about 0.05 wt. %, about 0.1 wt. %, about 0.2 wt. %, about 0.4 wt. %, about 0.6 wt. %, about 0.8 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, 19.0 wt. %, about 19.5 wt. %, or about 20.0 wt. %, including any values or ranges therebetween, of the KM-001 presented in the applied composition in the skin into stratum corneum, epidermis, or dermis. In embodiments, the topical administration to the skin of a patient in need thereof provides penetration of about 1 wt. % to about 10 wt. % of the KM-001 presented in the applied composition in the skin into the stratum corneum, as measured by IVPT. In embodiments, the topical administration to the skin of a patient in need thereof provides penetration of about 0.2 wt. % to about 8 wt. % of the KM-001 presented in the applied composition in the skin into the epidermis, as measured by IVPT. In embodiments, the topical administration to the skin of a patient in need thereof provides penetration of about 0.05 wt. % to about 3 wt. % of the KM-001 presented in the applied composition in the skin into the dermis, as measured by IVPT.

In embodiments, topical administration of the cream to the skin of a patient in need thereof provides penetration of about 50 μg/g to about 6000 μg/g, for example, about 50 μg/g, about 60 μg/g, about 70 μg/g, about 80 μg/g, about 90 μg/g, about 100 μg/g, about 150 μg/g, about 200 μg/g, about 250 μg/g, about 300 μg/g, about 350 μg/g, about 400 μg/g, about 450 g/g, about 500 μg/g, about 550 μg/g, about 600 μg/g, about 650 μg/g, about 700 μg/g, about 750 μg/g, about 800 μg/g, about 850 μg/g, about 900 μg/g, about 950 μg/g, about 1000 μg/g, about 1100 μg/g, about 1200 μg/g, about 1300 μg/g, about 1400 μg/g, about 1500 μg/g, about 1600 μg/g, about 1700 μg/g, about 1800 μg/g, about 1900 μg/g, about 2000 μg/g, about 2100 g/g, about 2200 μg/g, about 2300 μg/g, about 2400 μg/g, about 2500 μg/g, about 2600 μg/g, about 2700 μg/g, about 2800 μg/g, about 2900 μg/g, about 3000 μg/g, about 3100 μg/g, about 3200 μg/g, about 3300 μg/g, about 3400 μg/g, about 3500 μg/g, about 3600 μg/g, about 3700 g/g, about 3800 μg/g, about 3900 μg/g, about 4000 μg/g, about 4100 μg/g, about 4200 μg/g, about 4300 μg/g, about 4400 μg/g, about 4500 μg/g, about 4600 μg/g, about 4700 μg/g, about 4800 μg/g, about 4900 μg/g, about 5000 μg/g, about 5100 μg/g, about 5200 μg/g, about 5300 g/g, about 5400 μg/g, about 5500 μg/g, about 5600 μg/g, about 5700 μg/g, about 5800 μg/g, about 5900 μg/g, or about 6000 μg/g, including any values or ranges therebetween, of the KM-001 presented in the applied composition of about 0.2 wt. % to about 3 wt. % (e.g., about 0.3 wt. % or about 1 wt. %) in the skin into entire skin section, as measured by (matrix-assisted laser desorption/ionization) MALDI. In embodiments, topical administration of the cream to the skin of a patient in need thereof provides penetration of about 50 μg/g to about 6000 μg/g of the KM-001 presented in the applied composition of 1 wt. % in the skin into entire skin section, as measured by MALDI. In embodiments, topical administration of the cream to the skin of a patient in need thereof provides penetration of about 2000 μg/g to about 3000 μg/g of the KM-001 presented in the applied composition of 1 wt. % in the skin into entire skin section, as measured by MALDI. In embodiments, topical administration of the cream to the skin of a patient in need thereof provides penetration of about 2000 μg/g to about 6000 μg/g of the KM-001 presented in the applied composition of 1% w/w in the skin into all layers of epidermis, and the suprabasal epidermis layers in particular, as measured by MALDI. In embodiments, topical administration of the cream to the skin of a patient in need thereof provides penetration of about 500 μg/g to about 800 μg/g of the KM-001 presented in the applied composition of 1 wt. % in the skin into dermis, as measured by MALDI. In embodiments, topical administration of the cream to the skin of a patient in need thereof provides penetration in the skin into dermis, to achieve a dermis concentration of about 1% to about 16% (e.g., about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13.0%, about 13.5%, about 14.0%, about 14.5%, about 15.0%, about 15.5%, or about 16.0%, including any values or ranges therebetween) of the KM-001 compared to the KM-001 presented in epidermis, as measured by MALDI. In embodiments, topical administration of the cream to the skin of a patient in need thereof provides penetration of about 100 μg/g to about 3500 μg/g of the KM-001 presented in the applied composition of 1 wt. % in the skin into hypodermis, as measured by MALDI. In embodiments, topical administration of the cream to the skin of a patient in need thereof provides penetration of about 50 μg/g to about 1200 μg/g of the KM-001 presented in the applied composition of 1 wt. % in the skin into hair follicles, as measured by MALDI.

In embodiments, topical administration of about 2 grams to about 4 grams (e.g., about 2 g, about 2.1 g, about 2.2 g, about 2.3 g, about 2.4 g, about 2.5 g, about 2.6 g, about 2.7 g, about 2.8 g, about 2.9 g, about 3.0 g, about 3.1 g, about 3.2 g, about 3.3 g, about 3.4 g, about 3.5 g, about 3.6 g, about 3.7 g, about 3.8 g, about 3.9 g, or about 4.0 g, including any values or ranges therebetween) of the cream comprising 1 wt. % of KM-001 to about 400 cm² to about 600 cm² (e.g., about 410 cm², about 420 cm², about 430 cm², about 440 cm², about 450 cm² about 460 cm², about 470 cm², about 480 cm², about 490 cm², about 500 cm², about 510 cm², about 520 cm², about 530 cm², about 540 cm², about 550 cm², about 560 cm², about 570 cm², about 580 cm², about 590 cm², or about 600 cm², including any values or ranges therebetween) of the skin of a patient in need thereof provides about 50 μg/g to about 6000 g/g of the KM-001, for example, about 50 μg/g, about 60 μg/g, about 70 μg/g, about 80 μg/g, about 90 μg/g, about 100 μg/g, about 150 μg/g, about 200 μg/g, about 250 μg/g, about 300 g/g, about 350 μg/g, about 400 μg/g, about 450 μg/g, about 500 μg/g, about 550 μg/g, about 600 μg/g, about 650 μg/g, about 700 μg/g, about 750 μg/g, about 800 μg/g, about 850 μg/g, about 900 μg/g, about 950 μg/g, about 1000 μg/g, about 1100 μg/g, about 1200 μg/g, about 1300 μg/g, about 1400 μg/g, about 1500 μg/g, about 1600 μg/g, about 1700 μg/g, about 1800 g/g, about 1900 μg/g, about 2000 μg/g, about 2100 μg/g, about 2200 μg/g, about 2300 μg/g, about 2400 μg/g, about 2500 μg/g, about 2600 μg/g, about 2700 μg/g, about 2800 μg/g, about 2900 μg/g, about 3000 μg/g, about 3100 μg/g, about 3200 μg/g, about 3300 μg/g, about 3400 g/g, about 3500 μg/g, about 3600 μg/g, about 3700 μg/g, about 3800 μg/g, about 3900 μg/g, about 4000 μg/g, about 4100 μg/g, about 4200 μg/g, about 4300 μg/g, about 4400 μg/g, about 4500 μg/g, about 4600 μg/g, about 4700 μg/g, about 4800 μg/g, about 4900 μg/g, about 5000 g/g, about 5100 μg/g, about 5200 μg/g, about 5300 μg/g, about 5400 μg/g, about 5500 μg/g, about 5600 μg/g, about 5700 μg/g, about 5800 μg/g, about 5900 μg/g, or about 6000 μg/g, including any values or ranges therebetween, in one or more skin layers, as measured by (matrix-assisted laser desorption/ionization) MALDI. In embodiments, topical administration of about 3 grams of the cream comprising 1 wt. % of KM-001 to about 500 cm² of the skin provides about 50 μg/g to about 6000 μg/g of the KM-001 in one or more skin layers, as measured by (matrix-assisted laser desorption/ionization) MALDI.

In embodiments, topical administration of about 2 grams to about 4 grams of the cream comprising 1 wt. % of KM-001 to about 400 cm² to about 600 cm² of the skin of a patient in need thereof provides about 2000 μg/g to about 6000 μg/g of the KM-001 in epidermis, as measured by (matrix-assisted laser desorption/ionization) MALDI. In embodiments, topical administration of about 3 grams of the cream comprising 1 wt. % of KM-001 to about 500 cm² of the skin of a patient in need thereof provides about 2000 μg/g to about 6000 μg/g of the KM-001 in epidermis, as measured by (matrix-assisted laser desorption/ionization) MALDI. In embodiments, topical administration of about 2 grams to about 4 grams of the cream comprising 1 wt. % of KM-001 to about 400 cm² to about 600 cm² of the skin of a patient in need thereof provides about 2000 μg/g to about 6000 μg/g of the KM-001 in stratum basale, stratum spinosum, stratum granulosum, stratum lucidum, stratum corneum, or a combination thereof, as measured by MALDI. In embodiments, topical administration of about 2 grams to about 4 grams of the cream comprising 1 wt. % of KM-001 to about 400 cm² to about 600 cm² of the skin of a patient in need thereof provides about 500 μg/g to about 800 μg/g of the KM-001 in dermis, as measured by MALDI. In embodiments, topical administration of about 3 grams of the cream comprising 1 wt. % of KM-001 to about 500 cm² of the skin of a patient in need thereof provides about 500 μg/g to about 800 μg/g of the KM-001 in dermis, as measured by MALDI. In embodiments, topical administration of about 2 grams to about 4 grams of the cream comprising 1 wt. % of KM-001 to about 400 cm² to about 600 cm² of the skin of a patient in need thereof provides KM-001 in dermis at a concentration equivalent to about 1% to about 16% (e.g., about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13.0%, about 13.5%, about 14.0%, about 14.5%, about 15.0%, about 15.5%, or about 16.0%, including any values or ranges therebetween) of the KM-001 in epidermis, as measured by MALDI. In embodiments, topical administration of about 2 grams to about 4 grams of the cream comprising 1 wt. % of KM-001 to about 400 cm² to about 600 cm² of the skin of a patient in need thereof provides about 100 μg/g to about 3500 μg/g of the KM-001 in hypodermis, as measured by MALDI. In embodiments, topical administration of about 2 grams to about 4 grams of the cream comprising 1 wt. % of KM-001 to about 400 cm² to about 600 cm² of the skin of a patient in need thereof provides about 50 μg/g to about 1200 μg/g of the KM-001 in hair follicles, as measured by MALDI.

It is well known that the skin is an effective barrier, and thus the active agent in a topical composition must be in a dissolved state, and small enough to penetrate. If the active agent crystallizes or precipitates from the composition prior to (or during) application, the available dose of the active agent would be reduced. Thus, it is critical to select solvents and other components/excipients of the composition that maintain the solubility (and chemical stability) of the active agent (e.g., KM-001) during shipping, storage, and use.

In embodiments, the induction time of a sample comprising 100 pbw of the oil phase and 1 pbw of KM-001 is at least 500 minutes as measured by RapidOxy testing at 120° C., 700 kPa initial pressure, and 2% ΔP using 3 to 5 g of the sample. In embodiments, the cream meets the A criteria of European Pharmacopeia 5.1.3 after being stored for 1 year at 25° C.±2° C. and 60% relative humidity+5% relative humidity. In embodiments, the cream meets the A criteria of European Pharmacopeia 5.1.3 after being stored for 6 months at 40° C.±2° C. and 75% relative humidity±5% relative humidity. In embodiments, the cream meets the acceptance criteria of USP<51> after being stored for 6 months at 40° C.±2° C. and 75% relative humidity±5% relative humidity. In embodiments, the solubility of KM-001 in the oil phase is about 10 wt. % to about 15 wt. % at 10° C. to 30° C.

In embodiments, the compositions of the present disclosure promote delivery of the TRPV3 inhibitor to the intended layer(s) of the skin in order to treat various dermatological conditions (e.g., skin disorders and/or pruritus as described herein) while minimizing systemic exposure to the TRPV3 inhibitor. Systemic exposure is undesirable as it would increase the possibility of off-target effects which could impact the tolerability or safety of the composition. In addition, any TRPV3 inhibitor which is distributed systemically would not be available in the target tissue, i.e., skin, to treat the intended skin disorder(s) and/or pruritus. The compositions of the present disclosure have low systemic exposure levels, as described by the maximum plasma concentration (Cₘₐₓ) and the area under the plasma drug concentration-time curve (AUC) values. For example, the compositions of the present disclosure have Cₘₐₓ values in humans for the TRPV3 inhibitor (e.g., KM-001) of less than about 40 ng/mL (measured after 28 days in patients treated topically with a daily dose of about 0.4 mg/kg*day of the TRPV3 inhibitor). In embodiments, the Cₘₐₓ value after 28 days is from undetectable to about 0.1 ng/mL, about 0.2 ng/mL, about 0.3 ng/mL, about 0.4 ng/mL, about 0.5 ng/mL, about 0.6 ng/mL, about 0.7 ng/mL, about 0.8 ng/mL, about 0.9 ng/mL, about 1.0 ng/mL, about 1.5 ng/mL, about 2.0 ng/mL, about 2.5 ng/mL, about 3.0 ng/mL, about 3.5 ng/mL, about 4.0 ng/mL, about 4.5 ng/mL, about 5.0 ng/mL, about 6.0 ng/mL, about 6.5 ng/mL, about 7.0 ng/mL, about 7.5 ng/mL, about 8.0 ng/mL, about 8.5 ng/mL, about 9.0 ng/mL, about 9.5 ng/mL, about 10.0 ng/mL, about 10.5 ng/mL, about 11.0 ng/mL, about 11.5 ng/mL, about 12.0 ng/mL, about 12.5 ng/mL, about 13.0 ng/mL, about 13.5 ng/mL, about 14.0 ng/mL, about 14.5 ng/mL, about 15.0 ng/mL, about 15.5 ng/mL, about 16.0 ng/mL, about 16.5 ng/mL, about 17.0 ng/mL, about 17.5 ng/mL, about 18.0 ng/mL, about 18.5 ng/mL, about 19.0 ng/mL, about 19.5 ng/mL, about 20.0 ng/mL, about 20.5 ng/mL, about 21.0 ng/mL, about 21.5 ng/mL, about 22.0 ng/mL, about 22.5 ng/mL, about 23.0 ng/mL, about 23.5 ng/mL, about 24.0 ng/mL, about 24.5 ng/mL, about 25.0 ng/mL, about 25.5 ng/mL, about 26.0 ng/mL, about 26.5 ng/mL, about 27.0 ng/mL, about 27.5 ng/mL, about 28.0 ng/mL, about 28.5 ng/mL, about 29.0 ng/mL, about 29.5 ng/mL, about 30.0 ng/mL, about 30.5 ng/mL, about 31.0 ng/mL, about 31.5 ng/mL, about 32.0 ng/mL, about 32.5 ng/mL, about 33.0 ng/mL, about 33.5 ng/mL, about 34.0 ng/mL, about 34.5 ng/mL, about 35.0 ng/mL, about 35.5 ng/mL, about 36.0 ng/mL, about 36.5 ng/mL, about 37.0 ng/mL, about 37.5 ng/mL, about 38.0 ng/mL, about 38.5 ng/mL, about 39.0 ng/mL, about 39.5 ng/mL, or about 40.0 ng/mL, including all ranges between any of these values. In embodiments, the topical administration of the compositions (e.g., creams) of the present disclosure provides less than about 40 ng/mL of the maximum plasma concentration (Cₘₐₓ) of KM-001.

In embodiments, the AUC of the TRPV3 inhibitor (e.g., KM-001) (measured after 28 days in patients treated topically with a daily dose of about 0.4 mg/kg*day of the TRPV3 inhibitor) is less than about 500 ng/mL*h. In embodiments, the AUC for the TRPV3 inhibitor measured under such conditions ranges from undetectable to about 1 ng/mL*h, about 5 ng/mL*h, about 10 ng/mL*h, about 15 ng/mL*h, about 20 ng/mL*h, about 25 ng/mL*h, about 30 ng/mL*h, about 35 ng/mL*h, about 40 ng/mL*h, about 45 ng/mL*h, about 50 ng/mL*h, about 55 ng/mL*h, about 60 ng/mL*h, about 65 ng/mL*h, about 70 ng/mL*h, about 75 ng/mL*h, about 80 ng/mL*h, about 85 ng/mL*h, about 90 ng/mL*h, about 95 ng/mL*h, about 100 ng/mL*h, about 150 ng/mL*h, about 200 ng/mL*h, about 250 ng/mL*h, about 300 ng/mL*h, about 350 ng/mL*h, about 400 ng/mL*h, about 450 ng/mL*h, or about 500 ng/mL*h, including all ranges between any of these values. In embodiments, the topical administration provides less than about 500 ng/mL*h of the area under the plasma drug concentration-tine curve (AUC) of KM-001.

### Methods for Treating Skin Disorders and Pruritus

The compositions of the present disclosure, comprising about 0.2 wt. % to about 2 wt. %, in embodiments particularly comprising about 0.3 wt. % or about 1.0 wt. % of a TRPV3 inhibitor (e.g., KM-001) can be applied to regions of the patient's skin affected by a skin disorder and/or pruritus at least once daily. In some embodiments, the compositions of the present disclosure, as described herein, can be applied as needed, or more than once daily, for example twice daily (e.g., morning and evening), three times daily (morning, mid-day, and evening), or four or more times daily. The amount applied to the skin of the patient can be adjusted based on the concentration of the TRPV3 inhibitor (e.g., KM-001) in the composition, the severity of the skin disorder and/or pruritus, and the degree of clinical response. For example, compositions with higher compositions of the TRPV3 inhibitor (e.g., about 1.0 wt. % KM-001) may be applied less frequently than compositions with lower concentrations of the TRPV3 inhibitor (e.g., about 0.3 wt. % KM-001). Likewise, if the severity of the skin disorder and/or pruritus is lower, the frequency of administration or concentration of TRPV3 inhibitor can be decreased. In embodiments, the compositions of the present invention can be administered prophylactically, e.g., to a patient with a predisposition to a skin disorder, so as to delay or prevent the formation of symptoms of a dermatologic condition (e.g., keratodermic lesions and/or pruritus)

The amount of the compositions of the present disclosure applied daily, as discussed herein, can vary based on the severity of disease, the surface area of the affected skin of the patient, and the frequency of application.

In embodiments, suitable doses of the TRPV3 inhibitor (e.g., KM-001) of the compositions of the present disclosure can be expressed as an amount of the TRPV3 inhibitor (e.g., KM-001) per kg of the patient's body weight, administered per day. Expressed in this manner, suitable daily doses of the TRPV3 inhibitor (e.g., KM-001) range from about 0.1 to about 1 mg/kg/day, including about 0.1 mg/kg/day, about 0.15 mg/kg/day, about 0.2 mg/kg/day, about 0.25 mg/kg/day, about 0.3 mg/kg/day, about 0.35 mg/kg/day, about 0.4 mg/kg/day, about 0.45 mg/kg/day, about 0.5 mg/kg/day, about 0.55 mg/kg/day, about 0.6 mg/kg/day, about 0.65 mg/kg/day, about 0.7 mg/kg/day, about 0.75 mg/kg/day, about 0.8 mg/kg/day, about 0.85 mg/kg/day, about 0.9 mg/kg/day, about 0.95 mg/kg/day, or about 1.0 mg/kg/day, including all ranges between any of these values.

In embodiments, suitable doses of the TRPV3 inhibitor (e.g., KM-001) of the compositions of the present disclosure can be expressed as an amount of the TRPV3 inhibitor (e.g., KM-001) administered per cm² of skin, per day. Expressed in this manner, suitable daily doses of the TRPV3 inhibitor (e.g., KM-001) range from about 10 μg/cm²/day to about 1000 μg/cm²/day, including about 10 μg/cm²/day, about 20 μg/cm²/day, about 30 μg/cm²/day, about 40 μg/cm²/day, about 50 μg/cm²/day, about 60 μg/cm²/day, about 70 μg/cm²/day, about 80 μg/cm²/day, about 90 μg/cm²/day, about 100 μg/cm²/day, about 150 μg/cm²/day, about 200 μg/cm²/day, about 250 μg/cm²/day, about 300 μg/cm²/day, about 350 μg/cm²/day, about 400 μg/cm²/day, about 450 μg/cm²/day, about 500 μg/cm²/day, about 550 μg/cm²/day, about 600 μg/cm²/day, about 650 μg/cm²/day, about 700 μg/cm²/day, about 750 μg/cm²/day, about 800 μg/cm²/day, about 850 μg/cm²/day, about 900 μg/cm²/day, about 950 μg/cm²/day, or about 1000 μg/cm²/day, including all ranges between any of these values.

The compositions of the present disclosure can be administered over a defined period of time (e.g., 1, 2, 3, 4, or more weeks) until the patient's dermatological condition and/or pruritus is resolved or sufficiently treated to allow treatment to be discontinued, at least temporarily. In some embodiments, the compositions of the present disclosure can be administered indefinitely to patients suffering from a chronic dermatological condition or pruritus. In embodiments, the dose of the TRPV3 inhibitor (e.g., KM-001) administered may be adjusted to provide an appropriate maintenance dose to prevent flare ups or to prevent recurrence of the dermatological condition and/or pruritus. Dose adjustments may be provided by changing the concentration of the TRPV3 inhibitor (e.g., KM-001) in the topical composition, applying the same concentration at a different daily frequency, by applying a different amount of the topical composition, or some combination of these adjustments. Accordingly, to reduce the applied daily dose of the TRPV3 inhibitor (e.g., KM-001), the patient can switch to a topical composition with a lower concentration of the TRPV3 inhibitor, apply the topical composition less frequently, applying a lower amount of the topical composition, or some combination of these methods. Similarly, the daily dose of the TRPV3 inhibitor (e.g., KM-001) can be increased by switching to a topical composition with a higher concentration of the TRPV3 inhibitor, applying the topical composition more frequently, applying a greater amount of the topical composition, or some combination of these methods.

In embodiments, the present disclosure provides methods of use thereof for treating various dermatological conditions described herein, as well as for treating symptoms such as pruritus associated with (e.g., caused by or exhibited with) such dermatological conditions.

The compositions and methods of the present disclosure are effective in reducing the symptoms of the various dermatological conditions described herein, for example in reducing the size and severity of dermatological lesions. For example, the reduction in symptoms can be measured using the Investigator's Global Assessment (IGA) of disease severity, using the IGA scoring chart shown in Table 9 in Example 6.

The compositions of the present disclosure, comprising at least one TRPV3 inhibitor described herein (e.g., KM-001) are suitable for treating various skin disorders. For example, skin disorders treatable by the compositions of the present disclosure include keratodermas. Keratodermas are characterized by marked thickening of the epidermis of the skin. Keratodermas can be classified in various ways, depending on whether the keratoderma is inherited or acquired, as well as the clinical features of the keratoderma: diffuse keratodermas affect most of the palms and soles; focal keratodermas mainly affect pressure areas; and punctate-type keratodermas result in tiny bumps on the palms and soles. Most often the abnormal skin involves only the palms and soles (non-transgradient palmoplantar keratoderma) but sometimes it extends to the top of the hands and feet as well (transgradient).

Specific types of keratodermas treatable by the compositions and methods of the present disclosure include, but are not limited to, diffuse hereditary palmoplantar keratodermas (e.g., Unna-Thost type (autosomal dominant), Vorner's type (autosomal dominant), Mal de Meleda type (autosomal dominant or recessive), Huriez syndrome (autosomal dominant), Olmsted syndrome (unknown inheritance pattern), Vohwinkel syndrome (autosomal dominant), PPK with sensorineural deafness (mitochondrial inheritance), Bart-Pumphrey syndrome (autosomal dominant), Hidrotic ectodermal dysplasia (autosomal dominant), Papillon-Lefevre syndrome (autosomal recessive), Palmoplantar keratoderma Nagashima type (autosomal recessive), and Diffuse palmoplantar keratoderma with woolly hair and arrythmogenic cardiomyopathy (autosomal recessive); focal hereditary palmoplantar keratodermas (e.g., Palmoplantar keratoderma striata/areata type (autosomal dominant), Hereditary painful callosities (autosomal dominant), Howel-Evans syndrome or tylosis (autosomal dominant), Richner-Hanhart syndrome (autosomal recessive), Pachyonychia congenita (autosomal dominant), and Striate palmoplantar keratoderma with woolly hair and dilated cardiomyopathy (autosomal recessive); punctate palmoplantar keratodermas (e.g., Punctate keratoderma (autosomal dominant), Filiform keratoderma (autosomal dominant), Marginal keratoderma (acrokeratoelastoidosis, autosomal dominant). Keratodermas treatable by the compositions and methods of the present disclosure also include acquired palmoplantar keratodermas, which may be focal or diffuse. Such acquired keratodermas may arise in association with a variety of different skin and internal conditions, such as an inflammatory skin condition (e.g., eczema or psoriasis), infections, medications and toxins, an internal cancer, a systemic inflammatory disease, circulation problems, or sun damage.

Ichthyosis is another class of skin disorders treatable by the compositions and methods of the present disclosure. Ichthyosis refers to a relatively uncommon group of skin disorders characterized by the presence of excessive amounts of dry surface scales which are persistently dry, thickened, and engendering a “fish scale” appearance to the skin. Ichthyosis is regarded as a disorder of keratinization or cornification, and it is due to abnormal epidermal differentiation or metabolism.

The ichthyosiform dermatoses may be classified according to clinical manifestations, genetic presentation, and histologic findings. There are at least 20 different types of ichthyoses. Some types are inherited at birth and other types are acquired during adulthood. Inherited types of ichthyoses may be congenital or have delayed onset. Ichthyosis vulgaris has an autosomal dominant inheritance, meaning an abnormal gene is inherited from a parent. Penetrance is 90%, and onset is delayed until at least three months of age. Recessive X-linked ichthyosis mainly affects males, who have a single X chromosome with the abnormal gene. Females are protected by usually having a normal second X chromosome. Onset may be congenital or delayed by up to 6 months. In autosomal recessive congenital ichthyosis one abnormal gene is inherited from each parent. Congenital ichthyosiform erythroderma (CIE) is a variant of autosomal recessive congenital ichthyosis (ARCI), a rare epidermal disease, characterized by fine, whitish scales on a background of erythematous skin over the whole body. Keratinopathic ichthyoses have recessive and dominant forms and present at birth with a collodion membrane. Harlequin ichthyosis is a rare and severe form of ichthyosis that results in hard, thickened armor-like plates of skin covering the entire body from birth. Harlequin ichthyosis is also called harlequin-type ichthyosis, and harlequin fetus. Lamellar ichthyosis is a rare genetic condition. Infants affected by lamellar ichthyosis are generally born with a shiny, waxy layer of skin (called a collodion membrane) that is typically shed within the first two weeks of life. The skin beneath the collodion membrane is red and scaly. Epidermolytic ichthyosis (EI) is a rare, genetic skin disorder. It becomes apparent at birth, or shortly after birth, with reddening, scaling, and severe blistering of the skin. Hyperkeratosis develops within months and worsens over time. Blister formation decreases but may still occur after skin trauma or during the summer months. The skin can be itchy and smelly and is prone to infection. Other features may include reduced sweating; nail abnormalities; and in severe cases, growth failure. Superficial epidermolytic ichthyosis (SEI), formerly known as Ichthyosis bullosa of Siemens (IBS), is a rare keratinization disorder with superficial peeling. Although hyperkeratotic, the skin is unusually fragile and has a tendency to shed the outer layers of the epidermis, producing localized denuded areas. Netherton syndrome (ichthyosis linearis circumflexa) is a rare hereditary disorder characterized by scaling skin, hair anomalies, increased susceptibility to atopic eczema (a skin condition that can result in dry, red and flaky skin), elevated IgE levels, and other related symptoms. Netherton syndrome is inherited as an autosomal recessive trait. Pachyonychia congenita (PC) is a rare group of autosomal dominant skin disorders that are caused by a mutation in one of five different keratin genes. Pachyonychia congenita is often associated with thickened toenails, plantar keratoderma, and plantar pain.

Other types of ichthyosis treatable by the compositions and methods of the present disclosure include, but are not limited to, Chanarin-Dorfman syndrome (neutral lipid storage disease), CHILD syndrome (unilateral hemidysplasia), Conradi-Hunermann syndrome (X-linked dominant chondrodysplasia punctata), Darier disease, epidermal nevi (ichthyosis hystrix, linear epidermal nevus), epidermolytic hyperkeratosis (EHK), erythrokeratodermia variabilis (EKV), Giroux-Barbeau syndrome, Hailey-Hailey disease (benign familial pemphigus), ichthyosis hystrix Curth-Macklin type, keratosis follicularis spinulosa decalvans, KID syndrome (keratitis, ichthyosis, deafness), multiple sulfatase deficiency, peeling skin syndrome, pityriasis rubra pilaris (PRP), Refsum's disease (phytanic acid storage disease), Rud's syndrome, Sjogren-Larsson syndrome, and Tay's syndrome (trichothiodystrophy, IBIDS syndrome).

In embodiments, the compositions and methods of the present disclosure can be used to treat keratodermas, lichen simplex chronicus, pruritic conditions and ichthyoses. Specific keratodermas treatable by the compositions and methods of the present disclosure include Mal de Meleda type keratodermas, Olmstead syndrome, Papillon-Lefevre syndrome, palmoplantar keratoderma Nagashima type, pachyonychia congenita, and punctate palmoplantar keratoderma. Specific ichthyoses treatable by the compositions and methods of the present disclosure include recessive X-linked ichthyosis and Harlequin ichthyosis.

The compositions and methods of the present disclosure may also be used to treat pruritus or itch. Pruritus or itch may be defined as an unpleasant sensation of the skin that provokes the urge to scratch. It is a characteristic feature of many skin diseases and an unusual sign of some systemic diseases. Pruritus may be localized or generalized and can occur as an acute or chronic condition. Itching lasting more than 6 weeks is termed chronic pruritus.

Itching or pruritus exhibited as a symptom or component of various disorders (e.g., (atopic eczema, dermatitis herpetiformis, lichen simplex chronicus, and nodular prurigo) can also be treated by the compositions and methods of the present invention. These conditions are only rarely diagnosed in the absence of pruritus/itch. Chronic itch associated with the following dermatological disorders is also treatable by the compositions and methods of the present disclosure, including (but not limited to) Dermatitis herpetiformis, Dermatomyositis, Pemphigoid, Sjögren's syndrome; genetic related itch: Darier's disease, Hailey-Hailey disease, Ichthyoses, Sjögren-Larsson syndrome; Infection and Infestation related itch: Arthropod reactions, Dermatophytosis Folliculitis, Impetigo and other bacterial infections, Insect bites, Pediculosis, Scabies, Viral; Inflammatory related itch: Asteatosis (dry skin), including aging and senile pruritus, Atopic eczema, Contact dermatitis (irritant, allergic), Drug reactions, “Invisible dermatoses”, Lichen planus, Lichen simplex chronicus, Mastocytosis (urticaria pigmentosa), Miliaria, Psoriasis, Scars, Urticaria; Neoplastic related itch: Cutaneous T-cell lymphoma or mycosis fungoides (especially Sezary syndrome), Cutaneous B-cell lymphoma, Leukemia cutis; Pregnancy related itch: Pemphigoid gestationis, Polymorphic eruption of pregnancy, and Prurigo gestationis. Select systemic causes of chronic pruritus treatable by the compositions and methods of the present disclosure may include: Endocrine and Metabolic Diseases, such as Chronic renal failure, Diabetes mellitus (may be localized to scalp), Hyperthyroidism, Hypothyroidism, Liver disease (with or without cholestasis), Malabsorption, Perimenopausal pruritus; Infectious Diseases such as Helminthosis, HIV infection, Parasitosis; Neoplastic and hematological diseases such as Hodgkin's disease, Iron deficiency, Leukemia, Non-Hodgkin's lymphoma, Multiple myeloma, Plasmacytoma, Polycythemia rubra vera; Visceral Neoplasms such as Carcinoid syndrome and Solid tumors of the cervix, prostate, or colon; Pregnancy related disorders such as Pruritus gravidarum (with or without cholestasis); Induced by drugs, such as, Allopurinol, Amiodarone, Angiotensin-converting enzyme inhibitors, Estrogen, Hydrochlorothiazide, Hydroxyethyl cellulose, Opioids, Simvastatin. Other causes of chronic pruritis may result from Neurologic disease (Abscess, Infarcts, Multiple sclerosis, Notalgia Paresthetica, Tumors) or Psychiatric disease (Anxiety disorders, Depression, Obsessive-compulsive disorder).

In embodiments, the compositions and methods of the present disclosure are suitable and effective for treating pruritus or itch, either as a treatment of the pruritic symptoms alone, or as a therapy for a skin disorder, such as a keratoderma or lichen simplex chronicus together with their associated pruritic symptoms.

### NUMBERED EMBODIMENTS

1. A cream, comprising:

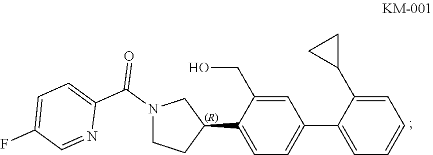

an aqueous phase; and
an oil phase,
wherein the concentration of KM-001 is about 0.1 wt. % to about 5.6 wt. %.
2. The cream of embodiment 1, wherein the concentration of KM-001 is about 0.2 wt. % to about 3 wt. %.
3. The cream of embodiment 1 or 2, wherein the concentration of KM-001 is about 0.3 wt. %.
4. The cream of embodiment 1 or 2, wherein the concentration of KM-001 is about 1 wt. %.
5. The cream of any one of embodiments 1-4, wherein the pH of the cream is about 4 to about 6.
6. The cream of any one of embodiments 1-5, wherein the pH of the cream is about 4.5 to about 5.5.
7. The cream of any one of embodiments 1-6, wherein the cream comprises:
   (a) about 20 wt. % to about 40 wt. % of the oil phase; and
   (b) about 60 wt. % to about 80 wt. % of the aqueous phase.
8. The cream of claim any one of embodiments 1-7, wherein the oil phase comprises triglyceride, C₁₃₋₂₁ fatty alcohol, or a mixture thereof.
9. The cream of embodiment 8, wherein the concentration of triglyceride is about 9 wt. % to about 13 wt. %.
10. The cream of embodiment 8 or 9, wherein the concentration of triglyceride is about 11 wt. %.
11. The cream of any one of embodiments 8-10, wherein the triglyceride is medium chain triglyceride (MCT).
12. The cream of embodiment 11, wherein the MCT is caprylic capric triglyceride.
13. The cream of any one of embodiments 8-12, wherein the concentration of C₁₃₋₂₁ fatty alcohol is about 10 wt. % to about 14 wt. %.
14. The cream of any one of embodiments 8-13, wherein the concentration of C₁₃₋₂₁ fatty alcohol is about 12 wt. %.
15. The cream of any one of embodiments 8-14, wherein the C₁₃₋₂₁ fatty alcohol is octyldodecanol.
16. The of claim any one of embodiments 1-15, wherein the oil phase is free of polyoxypropylene stearyl ether.
17. The cream of any one of embodiments 1-16, wherein the cream further comprises an antioxidant, a preservative, a viscosity modifying agent, a pH modifier, or a mixture thereof.
18. The cream of embodiment 17, wherein the antioxidant is propyl gallate, butylated hydroxyanisole (BHA), butylated hydroxytoluene, or a mixture thereof.
19. The cream of embodiment 18, wherein the antioxidant is a mixture of propyl gallate and BHA.
20. The cream of embodiment 19, wherein the concentration of propyl gallate is about 0.02 wt. % to about 0.08 wt. % and the concentration of BHA is about 0.05 wt. % to about 0.2 wt. %.
21. The cream of embodiment 20, wherein the cream comprises about 0.05 wt. % of propyl gallate and about 0.1 wt. % of BHA.
22. The cream of embodiment 17, wherein the concentration of preservative is about 0.05 wt. % to about 4 wt. %.
23. The cream of embodiment 22, wherein the preservative is a glycol ether, a phenol ether, a salt of benzoic acid, or a mixture thereof.
24. The cream of embodiment 23, wherein the preservative is about 0.05 wt. % to about 0.4 wt. % of phenoxyethanol, about 0.5 wt. % to about 2 wt. % of sodium benzoate, or a mixture thereof.
25. The cream of embodiment 17, wherein the viscosity modifying agent is a copolymer of acrylamide and sodium acryloyldimethyltaurate, polyoxyethylene sorbitan monooleate, sorbitan oleate, or a mixture thereof.
26. The cream of embodiment 25, wherein the viscosity modifying agent is about 1.5 wt. % to about 5 wt. % of the copolymer of acrylamide and sodium acryloyldimethyltaurate dispersed in isohexadecane, polyoxyethylene sorbitan monooleate, sorbitan oleate, or a mixture thereof.
27. The cream of embodiment 17, wherein the pH modifier is 10% citric acid.
28. The cream of any one of the preceding embodiments, wherein the cream further comprises glycerin, propylene glycol, or a mixture thereof.
29. The cream of any one of the preceding embodiments, wherein the viscosity of the cream is at least about 15,000 mPa·s, measured at 25° C. using a rotational viscosity method.
30. The cream of embodiment 29, wherein the viscosity of the cream is about 15,000 mPa·s to about 75,000 mPa·s, measured at 25° C. using a rotational viscosity method.
31. The cream of embodiment 29, wherein the viscosity of the cream is about 19,500 mPa·s to about 45,200 mPa·s, measured at 25° C. using a rotational viscosity method.
32. The cream of any one of the preceding embodiments, wherein the cream is an oil-in-water emulsion.
33. The cream of embodiment 32, wherein the mean diameter of globules of the emulsion is less than about 13 μm.
34. The cream of embodiment 32, wherein the mean diameter of globules of the emulsion is less than about 10 μm.
35. The cream of any one of the preceding embodiments, wherein the aqueous phase is a gel at about 2° C. to about 40° C.
36. The cream of embodiment 1, wherein topical administration of about 2 grams to about 4 grams of the cream comprising 1 wt. % of KM-001 to about 400 cm² to about 600 cm² of the skin provides about 50 μg/g to about 6000 μg/g of the KM-001 in one or more skin layers, as measured by (matrix-assisted laser desorption/ionization) MALDI.
36A. The cream of embodiment 36, wherein topical administration of about 3 grams of the cream comprising 1 wt. % of KM-001 to about 500 cm² of the skin provides about 50 μg/g to about 6000 μg/g of the KM-001 in one or more skin layers, as measured by (matrix-assisted laser desorption/ionization) MALDI
37. The cream of embodiment 36, wherein topical administration of about 2 grams to about 4 grams of the cream comprising 1 wt. % of KM-001 to about 400 cm² to about 600 cm² of the skin provides about 2000 μg/g to about 6000 μg/g of the KM-001 in epidermis, as measured by (matrix-assisted laser desorption/ionization) MALDI.
38. The cream of embodiment 36, wherein topical administration of about 2 grams to about 4 grams of the cream comprising 1 wt. % of KM-001 to about 400 cm² to about 600 cm² of the skin provides about 2000 μg/g to about 6000 μg/g of the KM-001 in stratum basale, stratum spinosum, stratum granulosum, stratum lucidum, stratum corneum, or a combination thereof, as measured by (matrix-assisted laser desorption/ionization) MALDI.
39. The cream of embodiment 36, wherein topical administration of about 2 grams to about 4 grams of the cream comprising 1 wt. % of KM-001 to about 400 cm² to about 600 cm² of the skin provides about 500 μg/g to about 800 μg/g of the KM-001 in dermis, as measured by (matrix-assisted laser desorption/ionization) MALDI.
40. The cream of embodiment 1, wherein topical administration of about 2 grams to about 4 grams of the cream comprising 1 wt. % of KM-001 to about 400 cm² to about 600 cm² of the skin of a patient in need thereof provides KM-001 in dermis at a concentration equivalent to about 1% to about 16% of the KM-001 in epidermis, as measured by MALDI.
41. The cream of embodiment 36, wherein topical administration of about 2 grams to about 4 grams of the cream comprising 1 wt. % of KM-001 to about 400 cm² to about 600 cm² of the skin provides about 100 μg/g to about 3500 μg/g of the KM-001 in hypodermis, as measured by (matrix-assisted laser desorption/ionization) MALDI.
42. The cream of embodiment 36, wherein topical administration of about 2 grams to about 4 grams of the cream comprising 1 wt. % of KM-001 to about 400 cm² to about 600 cm² of the skin provides about 50 μg/g to about 1200 μg/g of the KM-001 in hair follicles, as measured by (matrix-assisted laser desorption/ionization) MALDI.
43. The cream of any one of the preceding embodiments, wherein topical administration to the skin of a patient in need thereof provides penetration of about 0.05 wt. % to about 20 wt. % of the KM-001 presented in the applied composition in the skin into stratum corneum, epidermis, or dermis in the skin, as measured by in vitro penetration test (IVPT).
44. The cream of embodiment 43, wherein the topical administration to the skin of a patient in need thereof provides penetration of about 1 wt. % to about 10 wt. % of the KM-001 presented in the applied composition in the skin into the stratum corneum, as measured by IVPT.
45. The cream of embodiment 43, wherein the topical administration to the skin of a patient in need thereof provides penetration of about 0.2 wt. % to about 8 wt. % of the KM-001 presented in the applied composition in the skin into the epidermis, as measured by IVPT.
46. The cream of embodiment 43, wherein the topical administration to the skin of a patient in need thereof provides penetration of about 0.05 wt. % to about 3 wt. % of the KM-001 presented in the applied composition in the skin into the dermis, as measured by IVPT.
47. The cream of any one of the preceding embodiments, wherein the topical administration provides less than about 40 ng/mL of the maximum plasma concentration (Cₘₐₓ) of KM-001.
48. The cream of any one of the preceding embodiments, wherein the topical administration provides less than about 500 ng/mL*h of the area under the plasma drug concentration-tine curve (AUC) of KM-001.
49. A method of treating a skin disorder or pruritus, comprising topically administering a therapeutically effective amount of KM-001 in any one of the compositions of the preceding claims to the skin of a patient in need thereof.
50. The method of embodiment 49, wherein the KM-001 is administered at a daily dose of about 0.5 g to about 5 g.
51. The method of embodiment 49 or 50, wherein the therapeutically effective amount of KM-001 is 20 μg/cm²/day to about 150 μg/cm²/day.
52. The method of any one of embodiments 49-51, wherein the KM-001 is administered at a dosage of about 0.015 mg/kg/day to about 0.5 mg/kg/day.
53. The method of any one of embodiments 49-52, wherein the maximum plasma concentration (Cₘₐₓ) of KM-001 is less than about 40 ng/mL.
54. The method of any one of embodiments 49-53, wherein the area under the plasma drug concentration-tine curve (AUC) is less than about 500 ng/mL*h.
55. The method of any one of embodiments 49-54, wherein the skin disorder is keratodermas, lichen simplex chronicus, pruritus or ichthyoses.
56. The method of embodiment 55, wherein the skin disorder is a keratoderma selected from the group consisting of Mal de Meleda type keratodermas, Olmstead syndrome, Papillon-Lefevre syndrome, palmoplantar keratoderma, Palmoplantar keratoderma Nagashima type, pachyonychia congenita, and punctate keratoderma; and ichthyoses selected from the group consisting of recessive X-linked ichthyosis and Harlequin ichthyosis.
57. The method of embodiment 56, wherein the skin disorder is palmoplantar keratoderma.
58. The method of embodiment 57, wherein the palmoplantar keratoderma is punctate palmoplantar keratoderma or pachyonychia congenita.
59. The method of any one of embodiments 49-58, wherein the topical administration reduces the severity of the skin disorder at least 1 level of the IGA scale.
60. The method of any one of embodiments 49-59, wherein the pruritus is keratoderma associated pruritus.
61. The method of any one of embodiments 49-59, wherein the pruritus is lichen simplex chronicus associated pruritus.
62. The method of any one of embodiments 49-61, wherein the topical administration reduces severity of the pruritus by at least 1 level of the PP-NRS scale.
63. The method of embodiment 62, wherein the severity of the pruritus decreases about 1 level to about 10 levels of the PP-NRS scale.
64. The method of any one of embodiments 49-63, wherein the composition is administered twice daily.
65. The method of embodiment 64, wherein the composition is topically administered once in the morning and once in the evening.
66. The method of any one of embodiments 49-65, wherein the composition is topically administered for about 20 days to 40 days.
67. The method of any one of embodiments 49-66, wherein the composition is topically administered for about 28 days.
68. The method of any one of embodiments 49-67, wherein the composition is topically administered to a suprabasal layer of epidermis.
69. The method of embodiment 68, wherein the suprabasal layer of epidermis is cornified envelope, granular and spinous layer, upper dermis, or a combination thereof.
70. The method of embodiment 69, wherein the suprabasal layer of epidermis is cornified envelope, granular and spinous layer, and upper dermis.
71. The method of any one of embodiments 68-70, wherein the local concentration of KM-001 in the suprabasal layers of the epidermis is about 60 μg/g to about 160 mg/g following administration of the composition.
72. The method of any one of claims 68-70, wherein the local concentration of KM-001 in the upper dermis ranges from about 1.5 wt. % to about 16 wt. % of the total amount of KM-001 in the skin.

### EXAMPLES

### Example 1: Ointment and Cream-Type Emulsion Formulations

KM-001 is relatively soluble in triglycerides and fatty alcohols. Solubility studies showed that an oil phase containing about 11 wt. % medium chain triglycerides and 12 wt. % octyldodecanol allowed for complete and efficient solubilization of 1 wt. % KM-01 without any risk of recrystallization and provided physically stable emulsions. An ointment composition was formulated as shown in Table 1, below:

TABLE 1
PEG-based ointment (200293.0600)

| | | Composition |
|---|---|---|
| Ingredient | Chemical name | (% w/w) |
| Cos Tween 85-LQ-(CQ) | Polyoxyethylenesorbitan | 0.1 |
| | Trioleate | |
| benzyl alcohol | benzyl alcohol | 2 |
| Kollicream OA | oleyl alcohol | 5 |
| Transcutol HP | Diethylene glycol | 5 |
| | monoethyl ether | |
| Propylene carbonate | Propylene carbonate | 5 |
| 1,2-propanediol | 1,2-propanediol | 10 |
| Span 80-LQ-(MV) | sorbitan oleate | 1 |
| Kolliwax SA | stearyl alcohol | 5 |
| Super Refined PEG | polyethylene oxide, | 30 |
| 400-LQ-(LK) | molecular weight | |
| | 380-420 g/mol | |
| Kollisolv PEG 3350 | polyethylene oxide, | 35.9 |
| USP LAX | molecular weight | |
| | 3015-3685 g/mol | |
| KM-001 | (S)-2-(1,3-dimethyl-2-6- | 1 |
| | dioxo-2,3-1h-purin- | |
| | 7(6H)-N-(4-(3-Fluoro-4- | |
| | 2methylpyrrolidin-1- | |
| | yl)phenyl)thiazol-2- | |
| | yl)acetamide | |

Cream-type emulsions were found to have improved tolerability compared to the PEG-based ointment composition. In addition, the cream compositions did not require heating during formulation (as did the PEG-based ointment) which improved the stability of KM-001. Examples of three cream-type emulsions are found in Table 2, below.

TABLE 2
Cream-type Emulsions

| | | | Emulsion | Emulsion | Emulsion |
|---|---|---|---|---|---|
| | | | 200293.0435 | 200293.0440 | 200293.0438 |
| Ingredient | INCI name | Function | (% w/w) | (% w/w) | (% w/w) |
| KM-001 | N/A | API | 1.0 | 0.3 | 1.0 |
| sodium benzoate | sodium benzoate | preservative | 0.2 | 0.2 | 0.2 |
| phenoxy-2-ethanol | phenoxyethanol | preservative | 1.0 | 1.0 | 1.0 |
| glycerin | glycerin | hydrophilic vehicle | 2.0 | 2.0 | 2.0 |
| 1,2-propanediol | propylene glycol | solvent | 3.0 | 3.0 | 3.0 |
| propyl gallate | propyl gallate | antioxidant | 0.05 | 0.05 | 0.05 |
| butylated | butylated | antioxidant | 0.1 | 0.1 | 0.1 |
| hydroxyanisole | hydroxyanisole | | | | |
| Miglyol 812N | Medium chain | solvent | 11.0 | 11.0 | 8.0 |
| | Triglycerides | | | | |
| | (MCT): caprylic | | | | |
| | capric triglyceride | | | | |
| Kollicream OD | octyldodecanol | solvent | 12.0 | 12.0 | — |
| Polyoxypropylene | PPG-11 Stearyl | solvent | — | — | 15.0 |
| (11) Stearyl Ether | Ether | | | | |
| Sepineo P600 | acrylamide, AMPS | viscosity modifying | 3.0 | 3.0 | 3.0 |
| | copolymer dispersion | agent (stabilizer, | | | |
| | 40%/isohexadecane * | emulsifier) | | | |
| purified water | water | vehicle | Qs 100 | Qs 100 | Qs 100 |
| citric acid | citric acid | pH modifier | Qs pH = 4.5 | Qs pH = 4.5 | Qs pH = 4.5 |
| monohydrate | | | | | |

* alternative names: copolymer of acrylamide and sodium acryloyldimethyltaurate, isohexadecane, polysorbate 80, sorbitan oleate; Acrylamide/Sodium Acryloyldimethyltaurate Copolymer (and) Isohexadecane (and) Polysorbate 80

The 200293.0435, 200293.0440 and 200293.0600 formulations were tested using an in-vitro skin penetration test (IVPT) using split-thickness abdominal human skin having an approximate thickness of about 0.5 mm. Skin samples (approximately 2 cm²) were placed in a test cell using a PBS pH 7.2+0.25 Tween 80 receptor fluid and maintained at a temperature of 32+/−1° C., in a room maintained at 21.1±0.81° C., RH 48.13±16.1%. Skin samples were tested with a dose of 5 mg/cm² of the ointment and cream emulsion compositions over a 24-hour period, with receptor fluid samples collected at defined time points.

The samples were then prepared for subsequent chromatographic separation on a HPLC column and mass spectrometric detection. LC-MS/MS conditions are summarized below.

Column: Phenomenex: Kinetex C18 (2.6 μm, 50×2.1 mm)

Mobile Phase A: 1000 mL of water+0.195 formic acid

Mobile Phase B: 1000 mL Acetonitrile+0.10 formic acid

Retention times: KM-001 (˜0.91 min) and KM-001-D4 (˜0.90 min)

| | | | | |
|---|---|---|---|---|
| Gradient | Time | A | B | Flow rate |
| composition | (min) | (%) | (%) | (μL/min) |

| | | | |
|---|---|---|---|
| Initial | 45 | 55 | 500 |
| 1.00 | 5 | 95 | 500 |
| 2.00 | 5 | 95 | 500 |
| 2.01 | 45 | 55 | 500 |
| 4.00 | 45 | 55 | 500 |

Mass transitions (m/z): KM-001 (417.279 > 274.700; dwell 150 msec) and KM-001-D4 (421.286 > 278.800; dwell 150 msec).

The comparative penetration data of KM-001 into the skin layers such as stratum corneum, epidermis, and dermis, from the ointment and cream emulsion, is presented below:

TABLE 3
IVPT results: KM-001 accumulation in skin layers

| | PEG Ointment 1% | | Cream 0.3% | | Cream 1% | |
|---|---|---|---|---|---|---|
| | 200293.0600 | | 200293.0435 | | 200293.0440 | |
| | Accumulated | | Accumulated | | Accumulated | |
| | amount | | amount | | amount | |
| | (n = 6) | | (n = 9) | | (n = 9) | |
| Skin | | % of | | % of | | % of |
| Layers | ng | Dose | ng | Dose | ng | Dose |
| Stratum | 330.78 | 0.376 | 1503.12 | 4.67 | 4321.4 | 4.03 |
| Corneum | | | | | | |
| Epidermis | 563.22 | 0.64 | 885.78 | 2.75 | 3790.0 | 3.49 |
| Dermis | 57.21 | 0.065 | 141.58 | 0.43 | 430.5 | 0.40 |

Advanced emulsions, 200293.0435 and 200293.0440 were developed with several advantages over the preliminary PEG-based ointment and are highlighted below.

Emulsions 200293.0435 and 200293.0440 are classified as creams. These compositions utilized lipophilic excipients as primary solvents for KM-001. The concentrations of the lipophilic solvents enabled solubilization of KM-001 at an equivalent concentration to the PEG-based ointment but offered potential for enhanced local tolerance in combination with regulatory and pharmaceutical acceptance. The lipophilic solvents used in 200293.0435 and 200293.0440 generally exhibit enhanced local tolerance than the polar solvents used in the PEG-based ointment e.g., Transcutol HP, Propylene Carbonate, Propanediol-1.2 and Super Refined PEG 400.

Formulas 200293.0435 and 200293.0440 were able to dissolve the targeted amount of KM-001 while utilizing 3 times lower concentration of propylene glycol than the PEG-based ointment. This enabled retention of the beneficial effects of PG such as solubility for KM-001 and appropriate dermal delivery of KM-001 while reducing potential local tolerance issues that can be encountered with higher concentrations of glycols and other polar solvents.

Emulsions 200293.0435 and 200293.0440 also enabled the use of water as a vehicle which was considered advantageous from a tolerance perspective when compared with the anhydrous and polar solvent-loaded PEG-ointment.

The polymeric viscosity modifying agent, Sepioneo P600 stabilized the emulsion without the need of additional surfactants such as those used in the PEG-based ointment (Tween 85). This was also considered advantageous from a local tolerance perspective.

The cream formulations possess significant sensory benefits compared with the PEG-based ointment. The advanced cream prototypes provide a non-greasy and very pleasant texture that encourage and enhance patient treatment acceptance.

The cream formulas utilize a cold process that supports the chemical stability of KM-001. PEG ointment process required heat to melt certain components. A cold process may offer advantages from a scale-up perspective.

Formulas 200293.0435 and 200293.0440 contain pharmaceutical grade excipients that are compliant to United states Pharmacopeia (USP) or/and European Pharmacopoeia (Ph. Eur.) or listed in the FDAs IID. All raw materials in the emulsions were used at/or below concentrations in FDA approved topical products.

In vitro skin penetration testing (IVPT) showed improved delivery of KM-001 to the epidermis and dermis from the cream formulations comparing to the PEG-ointment formulation (see Table 3). These results were obtained using solvent systems in the emulsions that offered local tolerance advantages over the PEG-based ointment.

### Example 2: Evaluation of KM-001-Containing Formulations in a Minipig Animal Model

The PEG-based ointment described in Table 1 was found to have unstable physical properties, as over time it became grainy and hardened. In addition, some of the excipients are not currently approved by the appropriate regulatory bodies (e.g., US FDA) for use in human drug products. Accordingly, it was desirable to develop a more stable formulation with pharmaceutically acceptable excipients, with improved toxicity and dermal irritation properties.

Minipigs (Sus domesticus) are considered to be one of the major animal species used in translational research, surgical models, and procedural training are increasingly being used as an alternative to the dog or monkey as the choice of non-rodent species in preclinical toxicology testing of pharmaceuticals. There are unique advantages for the use of swine in this setting given that they share with humans similar anatomic and physiologic characteristics involving the cardiovascular, urinary, integumentary, and digestive systems [Swindle MM (ed.), Swine as Models in Biomedical Research and Toxicology Testing, Veterinary Pathology March 2012 vol. 49 no.2 344-356].

Four formulations were evaluated in minipigs as described below: 200293.0435 and 200293.0438, as well as placebo versions of these formulations, containing no KM-001. The minipigs were housed under a 12 hour light/dark cycle. The pigs' environment was controlled by a HVAC system (heating, ventilation, and air conditioning) at a temperature range of 16-27° C. and a relative humidity of 30-70% with adequate fresh air circulation. The test creams (KM-001 and placebo) were applied twice daily (except weekends, when the formulations were applied once daily), 3 g of formulation per pig, per area over 14 days.

The day prior to testing with creams containing KM-001, pigs received a prophylactic loading dose of antibiotic—Pen & Strep inj. susp. (1 mL/10 kg, IM) and Cefazoline HCl (30 mg/kg, IV) and marbofloxacin (2 mg/kg, slow IV), prior to cannulation (i.e., for blood sampling).

Procedure: KM-001 cream was applied on the 25×20 cm (500 cm²) shaved area on the animal's back. The tested skin area was covered. About 3 g per pig per area was used for each treatment. The treatments were repeated twice a day for 14 days.

After application of the KM-001 containing cream compositions, blood samples were taken at time 0 (baseline), 0.5 hr, 1 hr, 2 hrs, 6 hrs, and 24 hrs. Blood samples were also taken at time 0 (baseline), 0.5 hr, 1 hr, 2 hrs, 6 hrs, and 24 hrs after 14 days of application of the KM-001 containing creams. For comparative purposes, blood samples were also obtained from animals administered the placebo creams after 14 days of testing.

On scheduled termination day, 4 skin biopsies had been taken from the treated area of each animal, by means of 4 mm puncher. Of each set, three biopsies were subjected to snap-freeze, the other one to PFA 4% fixation for paraffin embedding. Full thickness punch biopsies were taken from the arbitrary three back sites at day 3 (end of the last day of the treatment) for assessment. Following a few days cleaning (wash-out), one additional back skin biopsy was taken to assess potential clearance. Biopsies were taken and subjected to snap-freezing from each animal without using tissue fixing or embedding reagents (e.g., OCT or paraffin).

Draize tests were performed on all animals twice each day when the cream formulations were applied (prior to each application and approximately 6 hours after application. The Draize scoring system used is provided below in Table 4.

TABLE 4
Draize Scoring System

| Score | Erythema | Edema |
|---|---|---|
| 0 | No erythema | No edema |
| 1 | Very slight erythema | Very slight edema |
| 2 | Well-defined erythema | Slight edema with raised margin |
| 3 | Moderate to severe | Moderate edema with raised |
| | erythema | margin ~1 mm |
| 4 | Severe erythema formation | Severe edema with raised |
| | | margin >1 mm and extending |
| | | beyond the area of exposure |

On the last day of testing, 4 skin biopsies were obtained from each test animal (placebo and KM-001 formulations). These skin samples were evaluated using quantitative mass spectrometry imaging (MALDI MSI) to obtain KM-001 biodistribution data, the KM-001 penetration profile, and absolute quantification of KM-001 within the minipig skin biopsy samples, to allow a comparison of two different KM-001 cream formulations (200293.0435 (F3) and 200293.0438 (F4)). Nine (9) minipig biopsies were evaluated (3 tissues treated with formulation F3, 3 tissues treated with formulation F4, and 3 placebo cream treated tissues). The results are provided below in Table 5.

TABLE 5
KM-001 Quantification (μg/g) Obtained by MALDI Imaging
in Control and Treated Minipig Skin Tissue Sections

| | Placebo | | |
|---|---|---|---|
| | (Avrg.) | KM-001 cream F3 | KM-001 cream F4 |
| Group | (μg/g) | (Avrg.) (μg/g) | (Avrg.) (μg/g) |
| Epidermis | n.d. | 2554.4 | 5261.1 |
| Dermis | n.d. | 539.3 | 882 |
| Hypodermis | n.d. | 127.8 | 3049 |
| Hair follicles | n.d. | 1093.7 | 54.5 |
| (total) | | | |
| Entire Section | n.d. | 2293.6 | 2367 |

MALDI imaging was carried out by the following method. Sectioning of minipig skin samples was performed at a thickness of 10 μm with a cryostat (HM 360, Microm) at the temperature of −21° C. The minipig skin sections were collected on Superfrost slide for both the MSI acquisitions and H&E (hematoxylin & eosin) staining then placed for 15 minutes in a desiccator before storage at −80° C. when they were not immediately used. KM-001 dilution series were prepared in water/methanol 50/50 (v/v). Each point was spotted on untreated minipig skin tissue sections provided by ImaBiotech (1 μL/drop deposit). The slides were then dried under vacuum for 15 min. DHB MALDI matrix at 40 mg/mL in methanol/water+1% TFA, 7:3 (v/v) spiked with 5 μM of KM-001-d4 was selected for the best detection of KM-001. This matrix was sprayed over the KM-001 dilution series spotted onto untreated minipig control skin sections provided by ImaBiotech and study samples with an automatic sprayer system (TM-Sprayer, HTX Imaging). MALDI-FTICR acquisition global parameters:
Mode: CASI
Ionization: positive
Mass ranges: 417±15 Da for KM-001 and KM-001-d4 analysis
Laser frequency: 2000 Hz
Spatial resolution: 200 μm for the calibration range of KM-001; 50 μm for the placebo and treated human skin tissue section.
Acquisition area:
   For calibration range of KM-001, each deposit of standard (17 concentrations) has been individually imaged.
   For samples, the entire minipig skin tissue section has been imaged by Mass Spectrometry Imaging All MSI acquisitions were performed with a data reduction at 0.95. The data reduction is required because of the large data generated by the MALDI when imaging regions.

The same MALDI method was used for all MSI acquisitions and the same mass spectrometer.

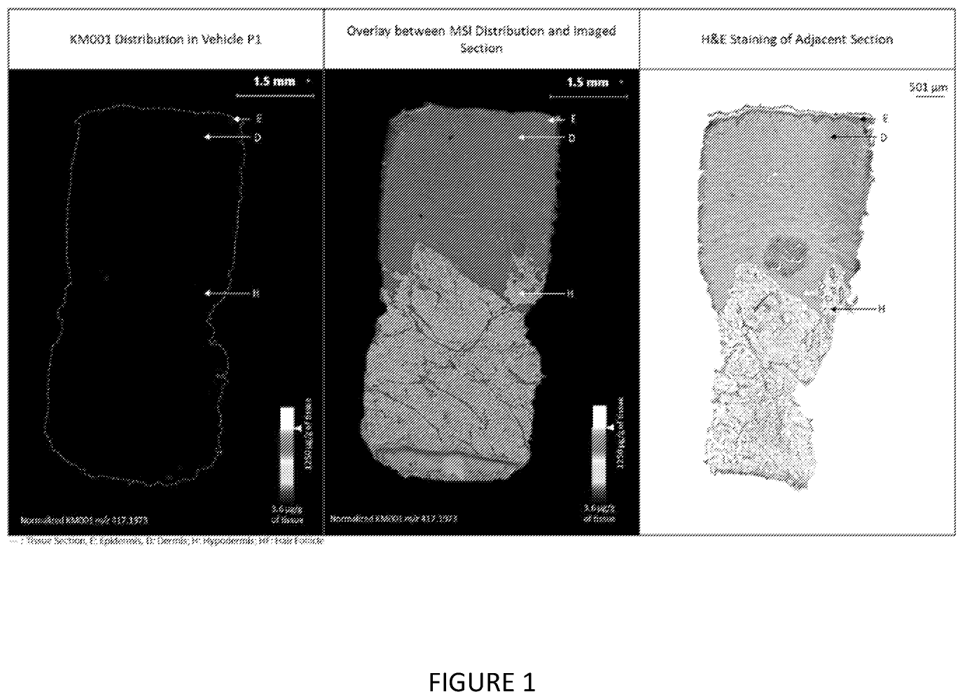

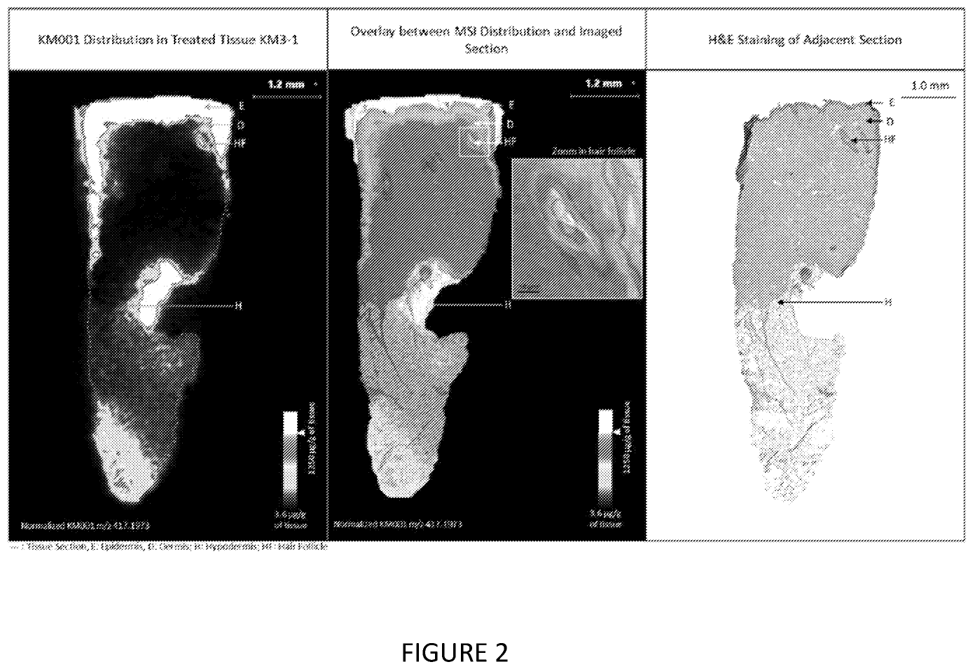

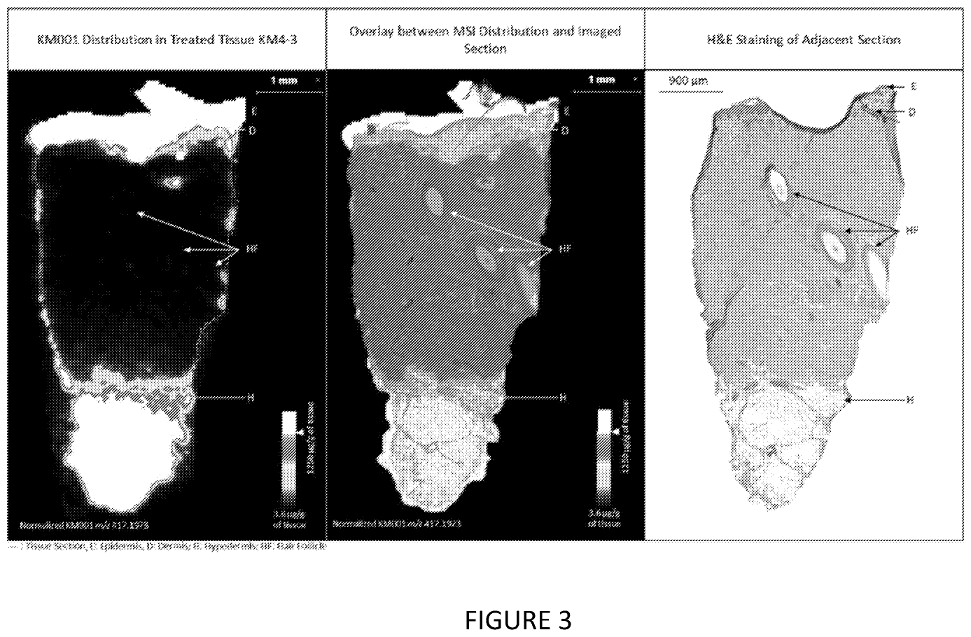

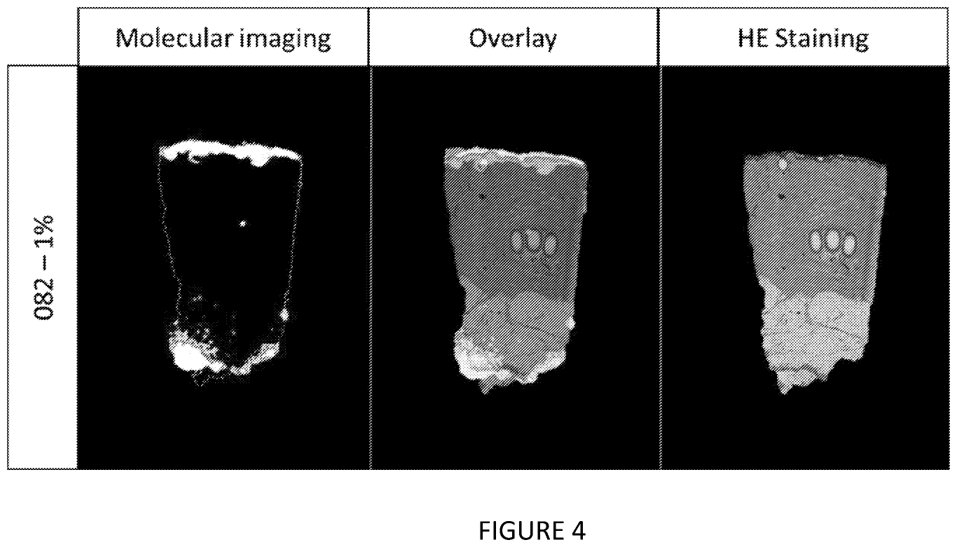

Nine (9) MALDI imaging acquisitions at a spatial resolution of 50 m were obtained using a CASI method in positive ion mode in order to image the KM-001 drug and its related internal standard (KM-001-d4) in six (6) treated tissues. Based on the distributions and the absolute quantification, penetration profiles of KM-001 were developed. The lower limit of quantification (LLOQ) and upper limit of quantification (ULOQ) were evaluated at 6.8 μg/g of tissue and 804.0 μg/g of tissue, respectively. KM-001 was mainly distributed in the epidermis, with high exposure (ULOQ—extrapolated between 67.3 μg/g and 5.7 mg/g for samples treated with the F3 formulation and between 2.6 and 147.3 mg/g for samples treated with the F4 formulation). Penetration was observed throughout all suprabasal layers of epidermis, including cornified envelopes, granular and spinous layers (indicated by yellow arrows in FIGS. 1-4).

Limited but marked penetration in the dermis (15.9% of the signal in epidermis was found in dermis for the F3 formulation samples; 1.5% for the F4 formulation samples). The hypodermis showed some exposure (117 μg/g in average for the 200293.0435 formulation samples and 3.5 mg/g in average for 200293.0438 formulation samples). On average (n=3), the cutaneous penetration seems to be slightly deeper in the KM-001 F4 formulation treatment group 4 compared to the F3 formulation treatment group (600 m versus 400 m).

As a comparison, the same study was performed when using the KM-001 ointment-based formulation (formulation 200293.0600). Imaging of the 0.3% KM-001 ointment and 1% KM-001 ointment-treated groups revealed limited penetration, mainly in the epidermis. Exposure of the uppermost layer of the epidermis was comparable to the F3 and F4 formulations, but no penetration was detected to the lower layers of epidermis or dermal penetration was observed for samples treated with the 200293.0600 ointment formulation.

Skin distribution of KM-001 in porcine biopsies in minipigs treated topically with PEG-ointment containing 200293.0600 1% w/w KM-001 was assessed. The MSI data showed strong exposure of the upper epidermis, low exposure of the dermis and limited exposure of the hypodermis to KM-001. While epidermis shows a rather homogeneous exposure, all along the epidermis, hypodermis is very heterogeneously exposed to KM-001, which is concentrated in specific regions. Calibration curves have been obtained and let to Lower Limit Of Quantification (LLOQ) of 4.8 μg/g of tissue and Upper Limit Of Quantification of 1052 μg/g.

Concentrations of KM-001 in the epidermis show an important variability [132-1104 μg/g] with the average values are 541 μg/g. In the dermis, concentrations are much lower, around 4 μg/g, e.g., close to the limit of quantification. In the hypodermis region, with an average concentration of 18 μg/g.

Finally, when considering the entire sections—the approach closest to homogeneization followed by LCMS/MS—the average concentrations of KM-001 of 26 μg/g were determined.

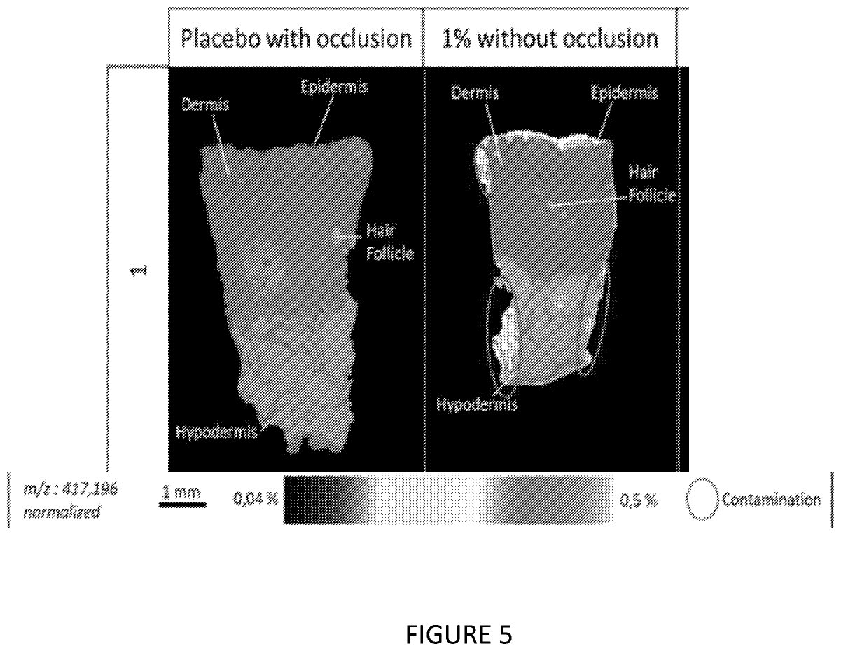

TABLE 6
KM-001 quantification (μg/g) obtained by MALDI Imaging
(FIG. 5) in control and treated porcine skin tissue sections:

| | Placebo (Avrg.) | KM-001 PEG ointment |
|---|---|---|
| Group | (μg/g) | (Avrg.) (μg/g) |
| Epidermis | n.d. | 541 |
| Dermis | n.d. | 4 |
| Hypodermis | n.d. | 6 |
| Entire Section | n.d. | 26 |

Thus, the KM-001 cream-based formulations (F3 and F4) show superior skin penetration profiles compared to the KM-001 ointment-based formulations (200293.0600), by delivering the KM-001 molecule to the site of action (i.e., all suprabasal epidermal layers), while also reaching the upper dermis (see Tables 5 and 6: 2554.4 μg/g (F3) and 5261.1 μg/g (F4) versus 541 μg/g (PEG-ointment) in epidermis; 539.3 μg/g (F3) and 882 μg/g (F4) versus 4 μg/g (PEG-ointment) in dermis).

### Example 3: Murine C57BL/6J Model (Oral Dosing)

The efficacy of KM-001 was assessed in an acetone-ether-water (AEW)-induced pruritus model in male C57BL/6J mice, a model induced by daily treatment of an acetone/ether (1:1) mixture and water applied to the animal's cheek. KM-001 (10 mg/kg, 1 mg/kg, 0.1 mg/kg and 0.01 mg/kg) was administered orally to 20 mice. The amount of time spent scratching and the number of bouts during the 20 minutes were recorded by an observer. Blood and skin samples were collected at 0, 0.5 hr, 1 hr and 1.5 hr post dosing.

Topical AEW application twice a day for 5 days significantly increased spontaneous scratching time and number of bouts in C57 BL/6J mice. Oral treatment with KM-001 (0.1 mg/kg) significantly decreased the scratching time and scratching bouts (p<0.001 in scratching time and p<0.01 scratching bouts). Treatment with KM-001 (0.01 mg/kg) trended to decrease scratching time and scratching bouts. In animals treated with KM-001 at 0.1 mg/kg, a maximum plasma concentration (Cₘₐₓ) of 1.34 ng/mL was detected at 0.5 h post-dosing reaching. In animals treated at 0.01 mg/kg, no KM-001 was detected in plasma. Results from this study indicated that KM-001 (0.1 mg/kg) has a robust anti-pruritic effect on AEW-induced dry skin pruritus model in mice.

### Example 4: Murine DS-Nh Model (Topical Dosing)

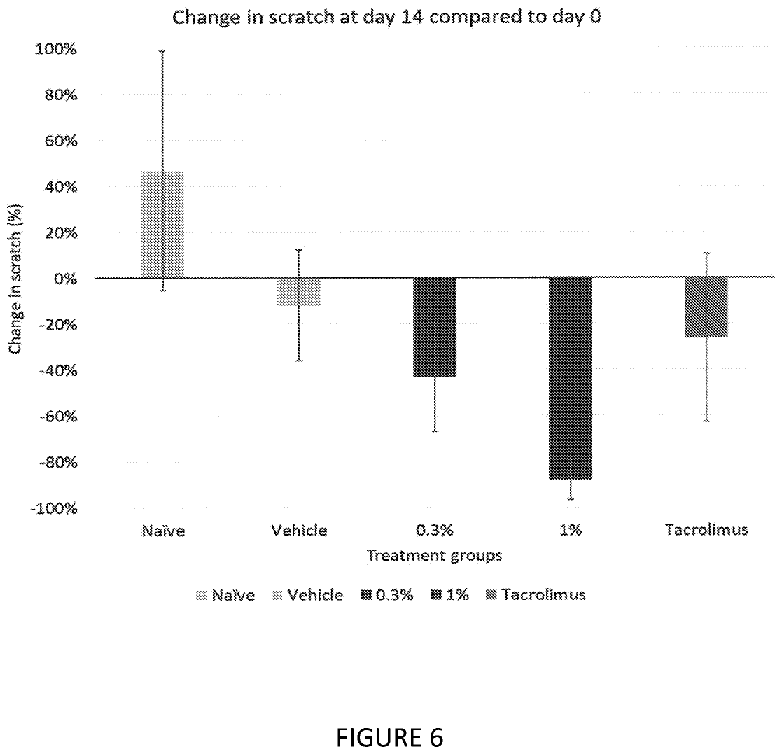

DS-Nh mice raised under conventional conditions spontaneously develop dermatitis and skin abnormalities characterized by hyperkeratosis, reduced basal keratinocytes proliferation, defective differentiation, and keratinization of skin appendages. DS/Nh mice treated twice daily (25 mg of ointment per cheek, one side per mouse) for 14 consecutive days, with KM-001 1% and 0.3% ointments (25 μg per cheek on one side/mouse) showed no compound-related toxicities. At baseline, marked scratching behavior was observed in all mice. Treatment with the vehicle and the positive control tacrolimus 0.1% only slightly affected this behavior (10% and 20% reduction, respectively). KM-001 0.3% ointment treatment resulted in a >40% reduction in scratch at day 14, and KM-001 1% ointment resulted in a scratch reduction of >80% in comparison to the naïve control. Changes in scratching behavior at day 14 vs. day 0 (prior to first application of experimental or control ointments) is shown in FIG. 6.

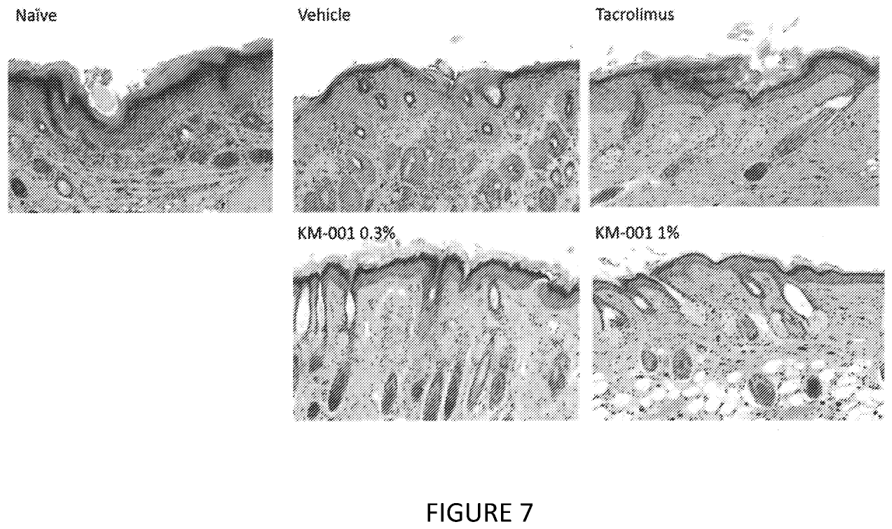

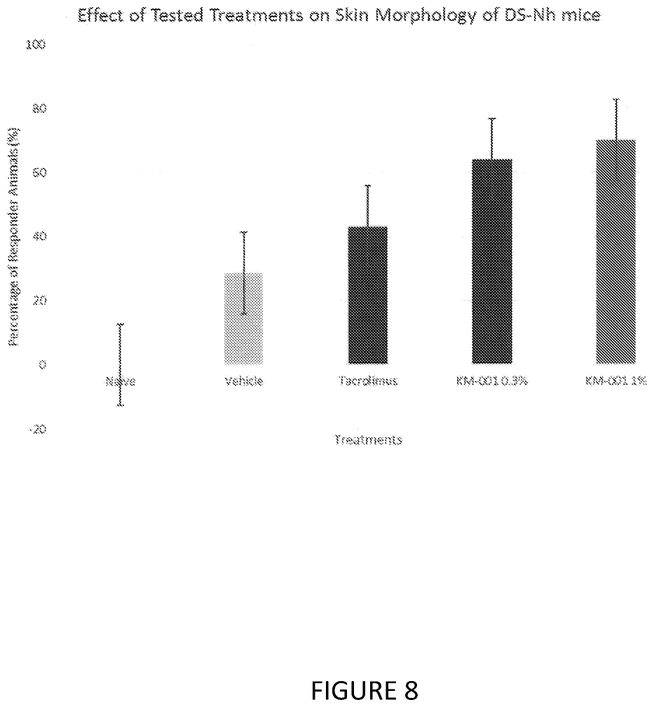

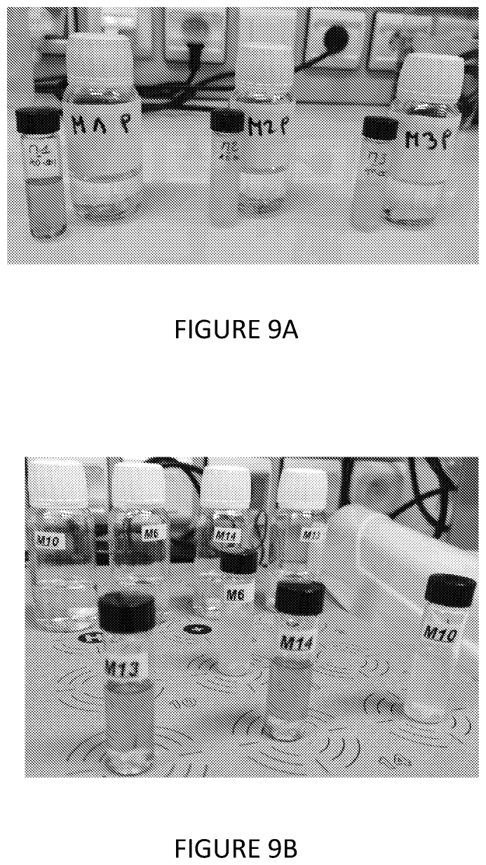

At the end of the study, the mice were sacrificed, and skin biopsies were paraffin-embedded, stained with hematoxylin and eosin (H&E) and assessed microscopically (FIG. 7). As expected, the skins of control mice exhibited characteristic pathology of thickened epidermis, abnormal epidermal layer structure, and fused and keratinized skin appendages. Following two weeks of treatment, only around 30% of mice in the vehicle-treated group showed some form of normalization of skin pathology, whereas the tacrolimus 0.1% treatment resulted in partial normalization in 40% of mice. In contrast, both KM-001 ointment-treated groups demonstrated striking improvements in skin histology, with around 70% of mice exhibiting normalization (FIG. 8). Animals treated with KM-001 1% ointment reached the 70% mark of improvement, alongside full skin normalization, which included restoration of hair follicles and sweat glands.

### Example 5: Systemic Exposure to KM-001 (Topical Administration)

I: Blood concentrations of KM-001 were evaluated using a validated analytical method as part of the toxicity studies in minipigs and rats. In minipigs, administration of 4.5 mg KM-001/kg/day gave Cₘₐₓ values of 10.8 ng/ml, and AUC values of 159.1 (ng/mL*h), respectively (Table 7). Based on a dose-linear extrapolation from minipig PK data after 13 weeks dermal administration and when considering the lower clinical dose (on a mg/kg basis), the human systemic exposure is estimated at a Cₘₐₓ of approx. 1.3 ng/mL (0.3% cream) and 4 ng/mL (1% cream) and an AUC of approx. 10 ng/mL*h (0.3%) and 32 ng/mL*h (1%) (Table 5). The systemic Cₘₐₓ in the 28-day oral repeat dose toxicity study in the rat at the NOAEL of 10 mg/kg/day was 968 ng/mL and for AUC 8357 ng/mL*h. Systemic exposure levels in the rat when compared to the estimated human systemic exposure after topical administration as planned for the human clinical study offers safety margins for Cₘₐₓ of approx. 242-fold (1% cream-gel); and for AUC safety margins would correspond to 836-fold (0.3%) and 261-fold (1%).

TABLE 7
Human and Animal Model Pharmacology

| | | KM-001 | | | | | | | | |
|---|---|---|---|---|---|---|---|---|---|---|
| | | Formulation | | API Dose | | | | Systemic | | Dose |
| | | Conc. | Dose | Level^{a} | HED^{b} | Cₘₐₓ^{c} | AUC₀₋ₜₗₐₛₜ^{d} | SF Rat | | MiniPig |
| Species | RoA | (%) | (g/day) | (mg/kg*day) | (mg/kg*day) | (ng/mL) | (ng/mL) | Cₘₐₓ | AUC | API |
| Human | dermal | 0.3 | 2 | 0.13 | — | (1.3)^{e} | (10)^{e} | 744 | 167 | 34 |
| | | 1.0 | 2 | 0.4 | — | (4) | (32)^{e} | 242 | 26 | 11 |
| MiniPig | dermal^{f} | 0.1 | 6 | 0.45 | N/A | 2.3 | 16.6 | — | — | — |
| | | 0.5 | 6 | 2.25 | N/A | 5.3 | 73.7 | — | — | — |
| | | 1.0 | 6 | 4.5 | N.A | 10.8 | 159.1 | — | — | — |
| Rat | oral | — | — | 1 | 0.16 | 97 | 479.4 | — | — | — |
| | | — | — | 5 | 0.81 | 525 | 4569 | — | — | — |
| | | — | — | 10 | 1.61 | 968 | 8357 | — | — | — |

^{a}Calculation based on mean body weight of animals from study (14 kg), and human body weight of 50 kg.
^{b}Rat conversion factor: 6.2; no conversion for dermal application in minipig applied.
^{c}Mean values for male and female rats (Day 28) and minipigs (Week 13).
^{d}Estimated human exposure based on minipig PK values, considering differences in the dose (mg/kg basis), and a conservative estimate of 2-fold higher human systemic exposure compared to minipig.
^{e}Assumed body surface area (BSA) treated: 10-60 cm².
^{f}Treated BSA: 500-600 cm².

II: The toxicokinetic profile of KM-001 cream, 0.3% w/w and 1% w/w in minipigs after twice daily dermal application for 2 weeks and recovery from any treatment-related effects during a recovery period of 1 week, was evaluated. Each test item was administered to the relevant group, by twice daily (at 6-hour intervals) topical application of 3 grams. Two additional groups were treated in the same manner with the control item, KM-001 Cream Placebo or reference item, KM-001 1% PEG Ointment.

In control animals, after a single and 14-day applications of KM-001 Cream Placebo, no detectable amount of KM-001 was found. After the first dose (Day 1), detectable amounts of KM-001, comparable or slightly higher than the lower limit of quantification, were measured in plasma of animals treated with test or reference items.

On Day 14, all minipigs were systemically exposed to KM-001 after repeated dermal treatment with test or reference items. Males and females treated with KM-001 0.3% Cream (Test item 1) and those receiving KM-001 1% PEG Ointment (Reference item) were similarly exposed to KM-001, while females treated with KM-001 1% Cream (Test item 2) appeared slightly more exposed than males. However, it should be noted that animals receiving KM-001 1% Cream were more exposed than those receiving Reference Item, containing the same concentration of KM-001. The exposure to KM-001 of the animals treated with Test items 1 and 2 increased with dose in a quite proportional manner. After 14-day of twice daily dermal application, plasma levels of KM-001 were still detectable 24 hours after treatment. Due to the behavior of plasma concentration along the last time point measured it was not possible to estimate the elimination phase of test item in a reliable manner.

TABLE 8
Mean blood toxicokinetic parameters for male and female minipigs on Day 14

| | Dose | | | Cmax | | AUC0- | | | | |
|---|---|---|---|---|---|---|---|---|---|---|
| Group | % | Cmax | | M/F | Tmax | tlast | | AUC0tlast | AUCinf | t1/2 |
| No./Sex | (mg/kg/day) | ng/mL | Cmax/dose | ratio | h | ng/mL*h | AUC0tlast/dose | M/F ratio | ng/mL*h | h |
| 0.3% cream | 0.3% | 8.178 | 6.340 | 1.237 | 1.667 | 128.6 | 99.72 | 1.109 | 394.8 | 41.95 |
| Males | (1.29) | | | | | | | | | |
| 0.3% cream | | 6.609 | 5.124 | | 4.833 | 116.0 | 89.9 | | 201.5 | 24.11 |
| Females | | | | | | | | | | |
| 1% cream | 1% | 22.24 | 5.173 | 0.740 | 1.667 | 353.2 | 82.13 | 0.751 | 864.6 | 32.77 |
| Males | (4.3) | | | | | | | | | |
| 1% cream | | 30.06 | 6.991 | | 1.417 | 470.5 | 109.4 | | 735.2 | 23.39 |
| Females | | | | | | | | | | |
| 1% ointment | 1% | 9.51 | 2.212 | 0.922 | 4 | 159.7 | 37.14 | 0.989 | 328.2 | 24.33 |
| Males | (4.3) | | | | | | | | | |
| 1% ointment | | 10.31 | 2.398 | | 0.750 | 161.5 | 37.55 | | 273.4 | 18.55 |
| Females | | | | | | | | | | |

### Example 6: Treatment of Punctate Keratoderma and Pachyonychia Congenita

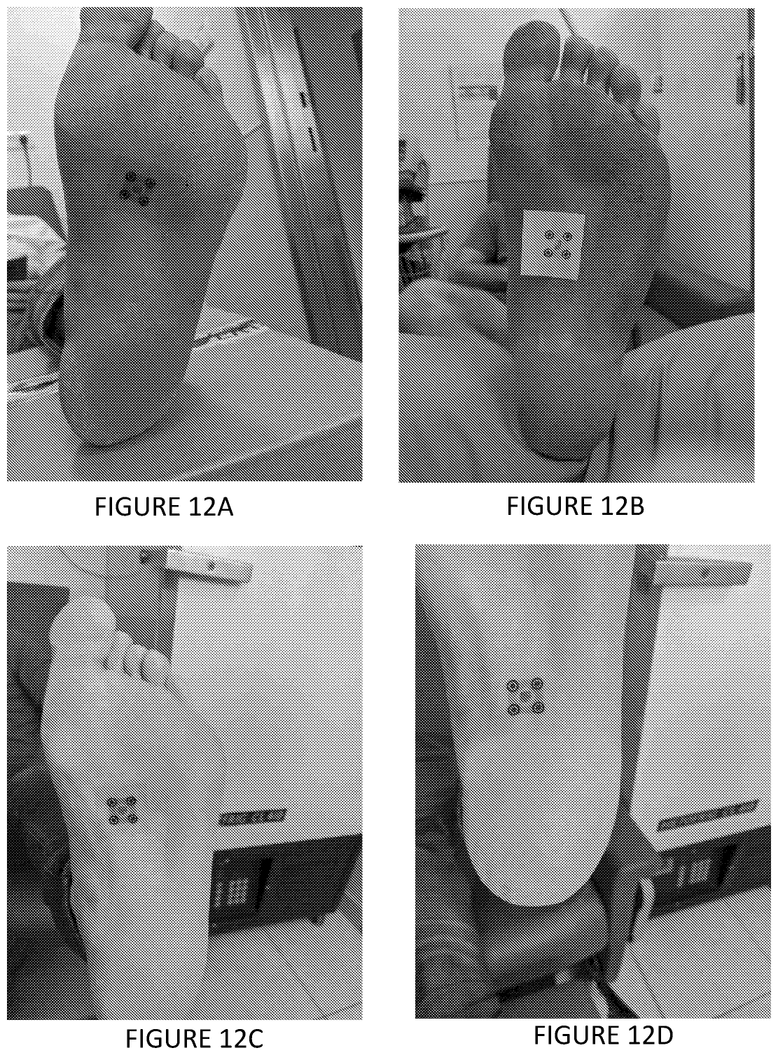

Patients with type 1 punctate palmoplantar keratoderma or pachyonychia congenita are treated with either 0.3% or 1% KM-001 cream twice daily in the affected areas of skin, for 84 consecutive days. Tolerability of the KM-001 composition and efficacy (as measured by improvement in the appearance of lesions) are evaluated using the IGA scoring method described below and in Table 9, prior to treatment with the compositions of the present disclosure, and then after treatment with the compositions of the present disclosure for a defined time period and dosing regimen. The IGA score is selected using the indicated descriptors that best describe the overall appearance of the patient's lesions at a given timepoint. It is not necessary that all characteristics under morphological description are present. Excoriations (i.e., abrasions or damage to the skin due to the patient's own picking, scratching, rubbing, etc.) are generally not considered when assessing disease severity. Changes in IGA score of one or more levels (e.g., 1, 2, 3 or 4 levels) indicate an improvement in the patient's condition, or alternatively stated, a reduction in disease severity. The compositions and methods of the present disclosure provide a reduction in disease severity of at least 1 level in the IGA score after treatment with the compositions of the present disclosure. Examples of an improvement of the affected feet area in Pachyonychia Congenita patients are shown in FIGS. 12A-D.

TABLE 9
IGA scoring

| Score | Level | Morphological Description |
|---|---|---|
| 0 | Clear | No inflammatory signs. Post-inflammatory |
| | | pigmentation may be present. |
| 1 | Almost | Barely perceptible erythema, barely perceptible |
| | clear | induration/papulation, and/or minimal |
| | | lichenification |
| 2 | Mild | Slight but definite erythema (pink), slight but |
| | | definite induration/papulation, and/or slight |
| | | but definite lichenification. |
| 3 | Moderate | Clearly perceptible erythema (dull red), clearly |
| | | perceptible induration/papulation, and/or clearly |
| | | perceptible lichenification. |
| 4 | Severe | Marked erythema (deep or bright red), marked |
| | | induration/papulation, and/or marked |
| | | lichenification. Disease is widespread in extent. |

### Example 7: Treatment of Pruritus in Patients with Lichen Simplex Chronicus

Patients with pruritus due to lichen simplex chronicus (LSC) are treated with either 0.3% or 1% KM-001 cream, or a negative control cream containing no KM-001. The patients' LSC lesions (lesions covering at least 0.5% of the patients' body skin area (BSA), up to 2% BSA) are treated with the KM-001 or control creams. Itch is assessed with the peak pruritus-numerical scale (PP-NRS; patients assess level of itch over the preceding 24 hour period using a 0-10 scale, where 0 is no itch and 10 is worst imaginable itch).

After treatment for at least 28 days, patients treated with the compositions of the present disclosure show improvements in LSC lesions as indicated by an improvement (decrease) in IGA scores, comparing lesion appearance pre- and post-treatment in individual patients. The mean IGA scores of patients treated with KM-001 compositions according to the present disclosure are statistically significantly lower than the IGA scores of patients treated with control formulations containing no KM-001.

After treatment for at least 28 days, patients treated with the compositions of the present disclosure show improvements in levels of itch, comparing PP-NRS scores pre- and post-treatment in individual patients. The mean PP-NRS scores of patients treated with KM-001 compositions according to the present disclosure are statistically significantly lower, e.g., by 4 or more points in the PP-NRS scale, compared to the PP-NRS scores of patients treated with control formulations containing no KM-001.

### Example 8: Batch Formula

Quantity per unit of each component in an exemplary batch formula of 1% KM-001 cream is provided below.

| | Quantity per | | Quantity per | |
|---|---|---|---|---|
| Component | unit (30 g) | | batch (6,400 g) | |
| KM-001 | 300 | mg | 64.0 | g |
| Sodium Benzoate | 60 | mg | 12.8 | g |
| Phenoxyethanol | 300 | mg | 64.0 | g |
| Glycerin | 600 | mg | 128.0 | g |
| Propylene Glycol | 900 | mg | 192.0 | g |
| Propyl Gallate | 15 | mg | 3.2 | g |
| Butylated Hydroxyanisole (BHA) | 30 | mg | 6.4 | g |
| Medium Chain Triglycerides (MCT)* | 3300 | mg | 704.0 | g |
| Octyldodecanol | 3600 | mg | 768.0 | g |
| Sepineo P600** | 900 | mg | 192.0 | g |

| | | |
|---|---|---|
| 10% w/w Citric Acid Solution | q.s. pH to 4.5 | q.s. pH to 4.5 |
| Purified Water | q.s. to 30 g | q.s. to 6,400 g |

### Example 9: Saturated Solubility of KM-001 in Selected Solvents Mixtures

Saturated solubility was determined using the shake flask method with excess API at room temperature (RT). It was assumed that equilibrium was attained after 24 hours, and excess solid was separated by ultrafiltration prior to dilution and quantification. Quantification of KM-001 for saturated solubility studies was performed using a qualified UPLC-UV analytical method.

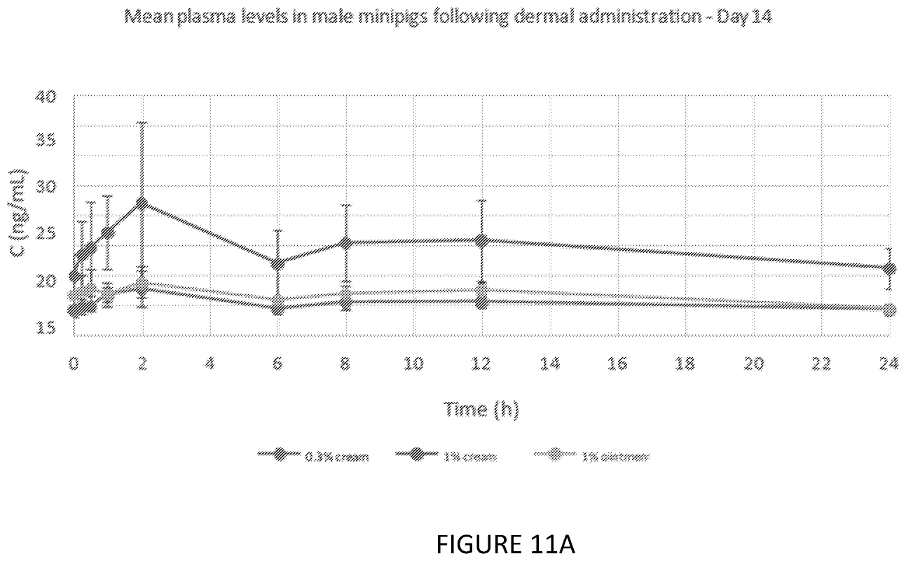

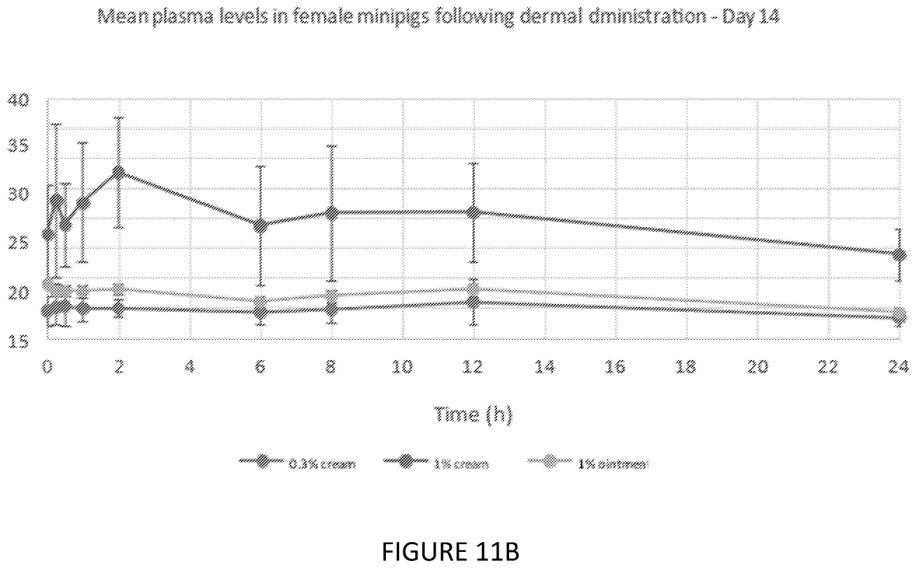

Saturated solubility of KM-001 in solvent mixtures is summarized in Table 10. Images of the samples after storage for 24 hours at 25° C. and their respective vehicles are presented in FIGS. 11A-B.

TABLE 10
Saturated solubility if KM-001 in solvent
mixtures as determined by UPLC-UV

| | | Excipients | Compo- | Saturated |
|---|---|---|---|---|
| Ref- | Excipients | INCI | sition | Solubility |
| erence | Trade name | Name | % | API (% w/w) |
| M1 | Miglyol 812 N | Medium Chain | 33.3 | 13.1 |
| | Arlamol E | Triglycerides | 62.5 | |
| | Phenoxy-2- | Polyoxypropylene | 4.2 | |
| | ethanol | Stearyl Ether | | |
| | | [symbol] | | |
| M2 | Miglyol 812 N | Medium Chain | 8 | 0.7 |
| | Arlamol E | Triglycerides | 15 | |
| | Macrol-82 | Polyoxypropylene | 77 | |
| | | Stearyl Ether | | |
| | | Light Mineral Oil | | |
| M3 | Miglyol 812 N | Medium Chain | 10 | 1.5 |
| | Kollicream OD | Triglycerides | 10 | |
| | Macrol-82 | Octyldodecanol | 75 | |
| | Kollicream OA | Light Mineral Oil | 5 | |
| | | Oleyl Alcohol | | |
| M6 | Miglyol 812 N | Medium Chain | 20 | 4.0 |
| | Arlamol E | Triglycerides | 16 | |
| | Kollicream OA | Polyoxypropylene | 10 | |
| | Macrol-82 | Stearyl Ether | 54 | |
| | | Oleyl Alcohol | | |
| M10 | Miglyol 812 N | Medium Chain | 20 | 1.6 |
| | Crodamol IPM- | Triglycerides | 21.85 | |
| | MBAL-LQ- | Isopropyl | 5 | |
| | (MV) | Myristate | | |
| | Kollicream OA | Oleyl Alcohol | 53.15 | |
| | | Light Mineral Oil | | |
| M13 | Miglyol 812 N | Medium Chain | 47.83 | 13.0 |
| | Kollicream OD | Triglycerides | 52.17 | |
| | | [symbol] | | |
| M14 | Miglyol 812 N | Medium Chain | 45.83 | 14.0 |
| | Kollicream OD | Triglycerides | 50 | |
| | Phenoxy-2- | Octyldodecanol | 4.17 | |
| | ethanol | Phenoxyethanol | | |

[symbol]

indicates data missing or illegible when filed

M1, M13 and M14 were designed for emulsion (cream-gel) concepts while M2, M3, M6 and M10 were designed to simulate petrolatum-based ointments. Mineral oil was used as a surrogate for petrolatum and other hydrocarbons that constitute the semi-solid (e.g., petrolatum) or solid (e.g., waxes) components of ointments. The mixtures containing Kollicream GD (Gctyldodecanol) or Arlamol E exhibited higher saturated solubility for KM-001. Model ointment blends containing “light mineral oil” exhibited lower saturated solubility. Results indicated that loading doses of up to 1% were theoretically possible in emulsions and ointment concepts. As shown in FIGS. 11A and 11B, the mixtures with higher KM-001 saturated solubility values exhibited more intense yellow color which was attributable to the API.

### Example 10: Evaluation of Antioxidant Blends with Model Oil Phases

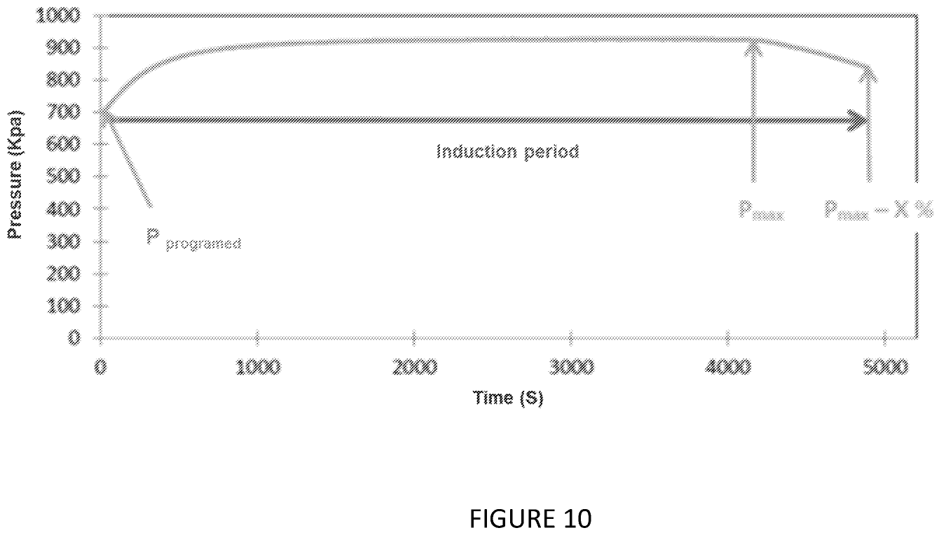

Screening efficacy of the antioxidant blends was conducted using the RapidGxy equipment. The RapidGxy methodology is based on the principle of O₂ consumption during oxidative reactions. The RapidOxy applies elevated pressure at high temperature for acceleration of the oxidation process and measures consumption of O₂ as a change in partial pressure. Induction time or induction period is defined as the time required to achieve a certain drop in O₂ partial pressure (FIG. 10). A typical RapidOxy testing protocol is described in Table 11.

TABLE 11
A typical RapidOxy testing protocol

| | | Selected significant | Quantity of |
|---|---|---|---|
| Temperature | Pressure | pressure variation ΔP | product |
| 120° C. | 700 kPa | 2% | 3 to 5 g |

RapidOxy methodology was utilized to assess the protective effect of pharmaceutical antioxidants on individual oils and model oil phases, prior to and after incorporation of KM-001. Individual antioxidants (Butylhydroxytoluene [Butylated Hydroxytoluene BHT], Butylhydroxyanisole [Butylated Hydroxyanisole, BHA] and Propyl gallate]) and antioxidant combinations with established synergy were also evaluated. Antioxidant combination #1 (AO1) incorporated 0.1% w/w Butylhydroxytoluene (Butylated Hydroxytoluene BHT) plus 0.1% w/w Butylhydroxyanisole (Butylated Hydroxyanisole, BHA). Antioxidant combination #2 (AO2) incorporated 0.1% w/w BHA plus 0.05% w/w Propyl gallate.

RapidOxy studies highlighted the oxidative sensitivity of some excipients, vehicle mixtures, KM-001 and the protective effects of various antioxidants. The results are summarized in Table 12.

Data indicated that Arlamol E was more sensitive to oxidation than Miglyol and that induction time decreased for both excipients when 1% KM-001 was added. Thus, Arlamol E was used as a model oxidation-sensitive solvent, rather than the more stable Miglyol, to evaluate the effect of individual antioxidants and antioxidant mixtures. The oxidative sensitivity of KM-001 was tested at 1% w/w in all experiments. Addition of KM-001 to Arlamol E resulted in substantial decrease of induction time indicating oxidative sensitivity of KM-001. The same tendency was observed also after addition of 1% KM-001 to Miglyol and RapOxM5 vehicle mixture. BHA 0.1% was more effective as single antioxidant comparing to BHT 0.1%, and Propyl gallate was the most efficient antioxidant when used alone.

Antioxidant combination AO2 (0.1% w/w BHA plus 0.05 w/w Propyl gallate) produced longer induction times, and this indicated that this antioxidant system was more effective than AO1 (0.1% w/w BHT+0.1% w/w BHA) and single antioxidants. The effectiveness of AO2 antioxidants' mixture was further confirmed in experiments with RapOxM5 vehicle mixture to which 10 of NKM-001 was added.

Studying oxidative sensitivity of vehicle mixtures revealed that Certain vehicle mixtures were more reactive to oxygen i.e., RapOxM3, RapOxM4 and RapOxM6 vehicles demonstrated significantly shorter induction times than RapOxM1, RapOxM2 and RapOxM5.

Conclusion: Screening antioxidant combinations in model systems using RapidOxy methodology demonstrated that mixture AO2 (BHA 0.1%, Propyl Gallate 0.050%) provided the most effective protection of the oils and active.

TABLE 12
Oxidative sensitivity screening of excipients, vehicle mixtures,
KM-001 and the protective effects of various antioxidants

| | | | | | | Induction |
|---|---|---|---|---|---|---|
| | Excipients | Excipients | Composition, | KM-001 | | Time, |
| Reference | Trade Name | INCI Name | % | Content | Antioxidants | minutes |
| RapOxAr1 | Arlamol E | Polyoxypropylene | Single | NA | NA | 418 |
| | | Stearyl Ether | excipient | | | |
| RapOxAr2 | Arlamol E | Polyoxypropylene | Single | 1% | NA | 227 |
| | | Stearyl Ether | excipient | | | |
| RapOxAr3 | Arlamol E | Polyoxypropylene | Single | 1% | BHT 0.1% | 302 |
| | | Stearyl Ether | excipient | | | |
| RapOxAr4 | Arlamol E | Polyoxypropylene | Single | 1% | BHA 0.1% | 482 |
| | | Stearyl Ether | excipient | | | |
| RapOxAr5 | Arlamol E | Polyoxypropylene | Single | 1% | AO1 | 486 |
| | | Stearyl Ether | excipient | | (BHA 0.1% + | |
| | | | | | BHT 0.1%) | |
| RapOxAr6 | Arlamol E | Polyoxypropylene | Single | 1% | Propyl | 561 |
| | | Stearyl Ether | excipient | | Gallate | |
| | | | | | 0.05% | |
| RapOxAr7 | Arlamol E | Polyoxypropylene | Single | 1% | AO2 | 612 |
| | | Stearyl Ether | excipient | | BHA 0.1% + | |
| | | | | | Propyl | |
| | | | | | Gallate | |
| | | | | | 0.05%) | |
| RapOxMig1 | Miglyol 812N | Medium Chain | Single | NA | NA | 700 |
| | | Triglycerides | excipient | | | |
| RapOxMig1 | Miglyol 812N | Medium Chain | Single | 1% | NA | 530 |
| | | Triglycerides | excipient | | | |
| RapOxM1 | Miglyol 812N | Medium Chain | 33.3% | NA | NA | 453 |
| | Arlamol E | Triglycerides | 62.5% | | | |
| | Phenoxyethanol | Polyoxypropylene | 4.2% | | | |
| | | Stearyl Ether | | | | |
| | | Phenoxyethanol | | | | |
| RapOxM2 | Miglyol 812N | Medium Chain | 8% | NA | NA | 588 |
| | Arlamol E | Triglycerides | 15% | | | |
| | Macrol-82 | Polyoxypropylene | 77% | | | |
| | | Stearyl Ether | | | | |
| | | Light Mineral Oil | | | | |
| RapOxM3 | Miglyol 812N | Medium Chain | 10% | NA | NA | 243 |
| | Kollicream OD | Triglycerides | 10% | | | |
| | Macrol-82 | Octyldodecanol | 75% | | | |
| | Kollicream OA | Light Mineral Oil | 5% | | | |
| | | Oleyl Alcohol | | | | |
| RapOxM4 | Miglyol 812N | Medium Chain | 10% | NA | NA | 127 |
| | Kollicream OD | Triglycerides | 10% | | | |
| | Macrol-82 | Polyoxypropylene | 80% | | | |
| | | Stearyl Ether | | | | |
| | | Light Mineral Oil | | | | |
| RapOxM5 | Miglyol 812N | Medium Chain | 20% | NA | NA | 588 |
| | Arlamol E | Triglycerides | 16% | | | |
| | Macrol-82 | Polyoxypropylene | 59% | | | |
| | Kollicream OA | Stearyl Ether | 5% | | | |
| | | Light Mineral Oil | | | | |
| | | Oleyl Alcohol | | | | |
| RapOxM5 | Miglyol 812N | Medium Chain | 20% | 1% | NA | 379 |
| | Arlamol E | Triglycerides | 16% | | | |
| | Macrol-82 | Polyoxypropylene | 59% | | | |
| | Kollicream OA | Stearyl Ether | 5% | | | |
| | | Light Mineral Oil | | | | |
| | | Oleyl Alcohol | | | | |
| RapOxM5 | Miglyol 812N | Medium Chain | 20% | 1% | AO2 | 651 |
| | Arlamol E | Triglycerides | 16% | | (BHA 0.1% + | |
| | Macrol-82 | Polyoxypropylene | 59% | | Propyl | |
| | Kollicream OA | Stearyl Ether | 5% | | Gallate | |
| | | Light Mineral Oil | | | 0.05%) | |
| | | Oleyl Alcohol | | | | |
| RapOxM6 | Miglyol 812N | Medium Chain | 20% | NA | NA | 293 |
| | Arlamol E | Triglycerides | 16% | | | |
| | Macrol-82 | Polyoxypropylene | 54% | | | |
| | Kollicream OA | Stearyl Ether | 10% | | | |
| | | Light Mineral Oil | | | | |
| | | Oleyl Alcohol | | | | |

### Example 11: Evaluation of Preservatives Efficacy of the Candidate Antimicrobial Preservatives Systems

Screening preservative efficacy test (PET) was conducted on samples stored for one-month at RT. Cream gel concept prototypes TD and composition are presented in Table 13 and petrolatum-based ointment prototypes TD and composition are presented in Table 14.

Two different antimicrobial preservative systems were evaluated for cream gel concept prototypes: (a) 0.2% Sodium Benzoate+1% Phenoxyethanol; and (b) 0.02% Propyl Paraben+0.18% Methyl paraben+100 Phenoxyethanol. The petrolatum-based ointment prototypes did not include antimicrobial preservatives since they are anhydrous systems and thus theoretically might possess self-preserving properties.

TABLE 13
Compositions of the cream-gels protypes selected for conducting Preservative efficacy testing (PET)

| | Solvent blend | | | | | |
|---|---|---|---|---|---|---|
| | M1 | | | | M13 | |
| | Containing Arlamol E | | | | Arlamol E free | |
| | Formula number | | | | | |
| | 200293.0436 | 200293.0437 | 200293.0438 | 200293.0439 | 200293.0435 | 200293.0440 |
| Ingredients | % w/w | | | | | |
| KM-001 | 1 | 0.3 | 1 | 0.3 | 1 | 0.3 |
| Propyl Gallate | 0.05 | 0.05 | 0.05 | 0.05 | 0.05 | 0.05 |
| Butylhydroxyanisole | 0.1 | 0.1 | 0.1 | 0.1 | 0.1 | 0.1 |
| Medium-chain | 8 | 8 | 8 | 8 | 11 | 11 |
| triglycerides/ | | | | | | |
| Miglyol 812N | | | | | | |
| Polyoxypropylene | 15 | 15 | 15 | 15 | / | / |
| stearyl ether/ | | | | | | |
| Arlamol PS11E-LQ | | | | | | |
| Octyldodecanol/ | / | / | / | / | 12 | 12 |
| Kollicream OD | | | | | | |
| Phenoxyethanol | 1 | 1 | 1 | 1 | 1 | 1 |
| Purified Water | qs100 | qs100 | qs100 | qs100 | qs100 | qs100 |
| Glycerol | 2 | 2 | 2 | 2 | 2 | 2 |
| Propylene Glycol | 3 | 3 | 3 | 3 | 3 | 3 |
| Methyl Paraben | 0.18 | 0.18 | / | / | / | / |
| Propyl Paraben | 0.02 | 0.02 | / | / | / | / |
| Sodium Benzoate | / | / | 0.2 | 0.2 | 0.2 | 0.2 |
| Sodium | Qs | Qs | / | / | / | / |
| Hydroxyde | pH = 5 ± 0.3 | pH = 5 ± 0.3 | | | | |
| Citric Acid | / | / | Qs | Qs | Qs | Qs |
| | | | pH = 4.5 ± 0.3 | pH = 4.5 ± 0.3 | pH = 4.5 ± 0.3 | pH = 4.5 ± 0.3 |
| Sepineo P600 | 3 | 3 | 3 | 3 | 3 | 3 |

TABLE 14
Compositions of the ointment protypes selected for
conducting Preservative efficacy testing (PET)

| | Solvent Blend | |
|---|---|---|
| | M6 variations with Arlamol E | |
| | formula number | |
| | 200293.0027 | 200293.0034 |
| Ingredients | % w/w | |
| KM-001 | 1 | 0.3 |
| Propyl Gallate | 0.05 | 0.05 |
| Butylhydroxyanisole | 0.1 | 0.1 |
| Oleyl alcohol/Kollicream OA | 10 | 10 |
| Polyoxypropylene stearyl ether/Arlamol | 16 | 16 |
| Medium-chain triglycerides/Miglyol 812N | 20 | 20 |
| Hydrogenated castor oil/Kolliwax HCO | 2 | 2 |
| Beeswax | 10 | 10 |
| Light mineral oil/Marcol 82 | 0.85 | 1.55 |
| Petrolatum/Vaseline Blanche Codex | 40 | 40 |

The PET was conducted according to the procedures described in respective chapters of Eur. Ph. and USP. The obtained results were assessed according to the acceptance criteria specified in European Pharmacopoeia 5.1.3 test criteria for topically used products provided in Table 15. The relevant acceptance criteria are Ph. Eur. Criteria A applied for common case of the preparations for cutaneous application.

TABLE 15
Ph. Eur. 5.1.3 Preservative Efficacy Tests Acceptance
Criteria for Preparations for Cutaneous Application

| | Ph. Eur. | Log Reduction | | | |
|---|---|---|---|---|---|
| Microorganisms | Criteria | 2 days | 7 days | 14 days | 28 days |
| Bacteria | A | ≥2 | ≥3 | — | NI |
| S. aureus - | B | — | — | ≥3 | NI |
| P. aeruginosa | | | | | |
| Yest and Mould | A | — | — | ≥2 | NI |
| C. albicans - | B | — | — | ≥1 | NI |
| A. brasiliensis | | | | | |

NI: no increase in number of viable micro-organisms compared to the previous reading.

The PET screening results for cream gel concept prototypes are presented in Table 16 and for petrolatum-based ointment prototypes are presented in Table 17.

TABLE 16
Logarithmic reduction compared to inoculum
- 28 days' results for cream-gel prototypes

| | Microorganisms | | | | | | | |
|---|---|---|---|---|---|---|---|---|
| | S. aureus | | P. aeruginosa | | C. albicans | | A. brasiliensis | |
| | ATCC 6538 | | ATCC 9027 | | ATCC 10231 | | ATCC 16404 | |
| | 14 | 28 | 14 | 28 | 14 | 28 | 14 | 28 |
| Formula | days | days | days | days | days | days | days | days |
| BP200293.0435 | >4.4 | >4.4 | >4.8 | >4.8 | >4.8 | >4.8 | 3.1 | >4.6 |
| BP200293.0436 | 2.1 | >4.4 | >4.8 | >4.8 | 0.6 | 0.7 | 0.9 | 2.0 |
| BP200293.0437 | 2.2 | >4.4 | >4.8 | >4.8 | 0.5 | 0.5 | 0.0 | 0.3 |
| BP200293.0438 | >4.4 | >4.4 | >4.8 | >4.8 | 1.4 | >4.8 | 1.5 | 2.1 |
| BP200293.0439 | >4.4 | >4.4 | >4.8 | >4.8 | 1.5 | >4.8 | 1.7 | 2.6 |
| BP200293.0440 | >4.4 | >4.4 | >4.8 | >4.8 | >4.8 | >4.8 | 3.4 | >4.6 |

(No data available at day 2 and day 7 for bacteria)

TABLE 17
Logarithmic reduction compared to inoculum - 28 days'
results for petrolatum-based ointment concepts.

| | Microorganisms | | | | | | | | | |
|---|---|---|---|---|---|---|---|---|---|---|
| | E. coli | | S. aureus | | P. aeruginosa | | C. albicans | | A. brasiliensis | |
| | ATCC 8739 | | ATCC 6538 | | ATCC 9027 | | ATCC 10231 | | ATCC 16404 | |
| | 14 | 28 | 14 | 28 | 14 | 28 | 14 | 28 | 14 | 28 |
| Formula | days | days | days | days | days | days | days | days | days | days |
| BP200293.0027 | >4.5 | >4.5 | >4.0 | >4.0 | >4.5 | >4.5 | >4.8 | >4.8 | −0.3 | 0.9 |
| BP200293.0034 | >4.5 | >4.5 | >4.0 | >4.0 | >4.5 | >4.5 | >4.8 | >4.8 | 0.0 | 0.4 |

(No data available at day 2 and day 7 for bacteria).

BP200293.0435 and BP200293.0440 prototypes of cream gel concept exhibited encouraging results of PET after storage for 1 month at RT and thus they were further evaluated after 3 months stability at accelerated storage conditions (40° C.). The efficacy of the antimicrobial preservative system of these samples, i.e., 0.2% Sodium Benzoate+1% Phenoxyethanol, was evaluated according to the to the acceptance Criteria A of Ph. Eur. 5.1.3 for preparations for cutaneous application and acceptance criteria of USP<51> specified for topically used products (Table 18).

TABLE 18
USP<51> Preservative Efficacy Tests Acceptance
Criteria Specified for Topically Used Products

| | Log Reduction | |
|---|---|---|
| Microorganisms | 14 days | 28 days |
| Bacteria | NLT 2.0 log reduction | No increase from the 14 |
| E. coli - S. aureus - | from the initial count | days' count at 28 days |
| P. aeruginosa | | |
| Yest and Mould | No increase from the | No increase from the |
| C. albicans - | initial calculated count | initial calculated count |
| A. brasiliensis | | |

“No increase” in counts is defined as NMT 0.5 log10 unit more than the value to which it is compared.

The results of PET of the BP200293.0435 and BP200293.0440 prototypes' samples stored for 3 months at accelerated conditions are presented in Table 19 and Table 20.

TABLE 19
Logarithmic reduction compared to inoculum - 28 days' results for cream
gels formula 200293.0435 and 200293.0440 as per usp <51>.

| | Microorganisms | | | | | | | | | |
|---|---|---|---|---|---|---|---|---|---|---|
| | E. coli | | S. aureus | | P. aeruginosa | | C. albicans | | A. brasiliensis | |
| | ATCC 8739 | | ATCC 6538 | | ATCC 9027 | | ATCC 10231 | | ATCC 16404 | |
| | 14 | 28 | 14 | 28 | 14 | 28 | 14 | 28 | 14 | 28 |
| Formula | days | days | days | days | days | days | days | days | days | days |
| 200293.0435 | >4.7 | >4.7 | >4.6 | >4.6 | >4.8 | >4.8 | >4.8 | >4.8 | 3.5 | >4.5 |
| 200293.0440 | >4.7 | >4.7 | >4.6 | >4.6 | >4.8 | >4.8 | >4.8 | >4.8 | >2.5 | >4.5 |

TABLE 20
Logarithmic reduction compared to inoculum - 28 days' results for
cream gels formula 200293.0435 and 200293.0440 as per Ph. Eur. (5.1.3.).

| | Microorganisms | | | | | | | | | | | |
|---|---|---|---|---|---|---|---|---|---|---|---|---|
| | S. aureus | | | | P. aeruginosa | | | | C. albicans | | A. brasiliensis | |
| | ATCC 6538 | | | | ATCC 9027 | | | | ATCC 10231 | | ATCC 16404 | |
| | 2 | 7 | 14 | 28 | 2 | 7 | 14 | 28 | 14 | 28 | 14 | 28 |
| Formula | days | days | days | days | days | days | days | days | days | days | days | days |
| 200293.0435 | >4.6 | >4.6 | >4.6 | >4.6 | >4.8 | >4.8 | >4.8 | >4.8 | >4.8 | >4.8 | 3.5 | >4.5 |
| 200293.0440 | >4.6 | >4.6 | >4.6 | >4.6 | >4.8 | >4.8 | >4.8 | >4.8 | >4.8 | >4.8 | >2.5 | >4.5 |

Both formulas, BP200293.0435 and BP200293.0440 passed USP acceptance criteria and Criteria A of European Pharmacopoeia. These tests verified preservative system efficacy data generated at the 1-month stability at RT and showed that 0.2% Sodium Benzoate+1% Phenoxyethanol is a suitable antimicrobial preservative system for BP200293.0435 and BP200293.0440 formulas.

1. A cream, comprising: 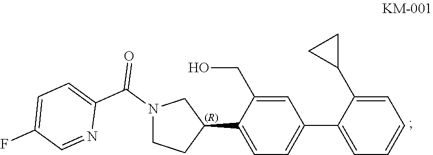
an aqueous phase; and
an oil phase,
wherein the concentration of KM-001 is about 0.1 wt. % to about 5.6 wt. %.

2. The cream of claim 1, wherein the concentration of KM-001 is about 0.2 wt. % to about 3 wt. %.

3. The cream of claim 1 or 2, wherein the concentration of KM-001 is about 0.3 wt. %.

4. The cream of claim 1 or 2, wherein the concentration of KM-001 is about 1 wt. %.

5. The cream of any one of claims 1-4, wherein the pH of the cream is about 4 to about 6.

6. The cream of any one of claims 1-5, wherein the pH of the cream is about 4.5 to about 5.5.

7. The cream of any one of claims 1-6, wherein the cream comprises:
(a) about 20 wt. % to about 40 wt. % of the oil phase; and
(b) about 60 wt. % to about 80 wt. % of the aqueous phase.

8. The cream of any one of claims 1-7, wherein the oil phase comprises triglyceride, C₁₃₋₂₁ fatty alcohol, or a mixture thereof.

9. The cream of claim 8, wherein the concentration of triglyceride is about 9 wt. % to about 13 wt. %.

10. The cream of claim 8 or 9, wherein the concentration of triglyceride is about 11 wt. %.

11. The cream of any one of claims 8-10, wherein the triglyceride is a medium chain triglyceride (MCT).

12. The cream of claim 11, wherein the MCT is caprylic capric triglyceride.

13. The cream of any one of claims 8-12, wherein the concentration of C₁₃₋₂₁ fatty alcohol is about 10 wt. % to about 14 wt. %.

14. The cream of any one of claims 8-13, wherein the concentration of C₁₃₋₂₁ fatty alcohol is about 12 wt. %.

15. The cream of any one of claims 8-14, wherein the C₁₃₋₂₁ fatty alcohol is octyldodecanol.

16. The cream of any one of claims 1-15, wherein the oil phase is free of polyoxypropylene stearyl ether.

17. The cream of any one of claims 1-16, wherein the cream further comprises an antioxidant, a preservative, a viscosity modifying agent, a pH modifier, or a mixture thereof.

18. The cream of claim 17, wherein the antioxidant is propyl gallate, butylated hydroxyanisole (BHA), butylated hydroxytoluene, or a mixture thereof.

19. The cream of claim 18, wherein the antioxidant comprises propyl gallate and BHA.

20. The cream of claim 19, wherein the concentration of propyl gallate is about 0.02 wt. % to about 0.08 wt. % and the concentration of BHA is about 0.05 wt. % to about 0.2 wt. %.

21. The cream of claim 20, wherein the cream comprises about 0.05 wt. % of propyl gallate and about 0.1 wt. % of BHA.

22. The cream of claim 17, wherein the concentration of the preservative is about 0.05 wt. % to about 4 wt. %.

23. The cream of claim 22, wherein the preservative is a glycol ether, a phenol ether, a salt of benzoic acid, or a mixture thereof.

24. The cream of claim 23, wherein the cream comprises about 0.05 wt. % to about 0.4 wt. % of phenoxyethanol, about 0.5 wt. % to about 2 wt. % of sodium benzoate, or a mixture thereof.

25. The cream of claim 17, wherein the viscosity modifying agent is a copolymer of acrylamide and sodium acryloyldimethyltaurate, polyoxyethylene sorbitan monooleate, sorbitan oleate, or a mixture thereof.

26. The cream of claim 25, wherein the viscosity modifying agent is about 1.5 wt. % to about 5 wt. % of a copolymer of acrylamide and sodium acryloyldimethyltaurate dispersed in isohexadecane, polyoxyethylene sorbitan monooleate, sorbitan oleate, or a mixture thereof.

27. The cream of claim 17, wherein the pH modifier is 10% citric acid.

28. The cream of any one of the preceding claims, wherein the cream further comprises glycerin, propylene glycol, or a mixture thereof.

29. The cream of any one of the preceding claims, wherein the viscosity of the cream is at least about 15,000 mPa·s, measured at 25° C. using a rotational viscosity method.

30. The cream of claim 29, wherein the viscosity of the cream is about 15,000 mPa·s to about 75,000 mPa·s, measured at 25° C. using a rotational viscosity method.

31. The cream of claim 29, wherein the viscosity of the cream is about 19,500 mPa·s to about 45,200 mPa·s, measured at 25° C. using a rotational viscosity method.

32. The cream of any one of the preceding claims, wherein the cream is an oil-in-water emulsion.

33. The cream of claim 32, wherein the mean diameter of globules of the emulsion is less than about 13 μm.

34. The cream of claim 33, wherein the mean diameter of globules of the emulsion is less than about 10 μm.

35. The cream of any one of the preceding claims, wherein the aqueous phase is a gel at about 2° C. to about 40° C.

36. The cream of claim 1, wherein topical administration of about 2 grams to about 4 grams of the cream comprising 1 wt. % of KM-001 to about 400 cm² to about 600 cm² of the skin of a patient in need thereof provides about 50 μg/g to about 6000 μg/g of the KM-001 in one or more skin layers, as measured by matrix-assisted laser desorption/ionization (MALDI).

37. The cream of claim 36, wherein topical administration of about 3 grams of the cream comprising 1 wt. % of KM-001 to about 500 cm² of the skin of a patient in need thereof provides about 50 μg/g to about 6000 μg/g of the KM-001 in one or more skin layers, as measured by MALDI.

38. The cream of claim 36, wherein topical administration of about 2 grams to about 4 grams of the cream comprising 1 wt. % of KM-001 to about 400 cm² to about 600 cm² of the skin of a patient in need thereof provides about 2000 μg/g to about 6000 μg/g of the KM-001 in epidermis, as measured by MALDI.

39. The cream of claim 36, wherein topical administration of about 2 grams to about 4 grams of the cream comprising 1 wt. % of KM-001 to about 400 cm² to about 600 cm² of the skin of a patient in need thereof provides about 2000 μg/g to about 6000 μg/g of the KM-001 in stratum basale, stratum spinosum, stratum granulosum, stratum lucidum, stratum corneum, or a combination thereof, as measured by MALDI.

40. The cream of claim 36, wherein topical administration of about 2 grams to about 4 grams of the cream comprising 1 wt. % of KM-001 to about 400 cm² to about 600 cm² of the skin of a patient in need thereof provides about 500 μg/g to about 800 μg/g of the KM-001 in dermis, as measured by MALDI.

41. The cream of claim 1, wherein topical administration of about 2 grams to about 4 grams of the cream comprising 1 wt. % of KM-001 to about 400 cm² to about 600 cm² of the skin of a patient in need thereof provides KM-001 in dermis at a concentration equivalent to about 1% to about 16% of the KM-001 in epidermis, as measured by MALDI.

42. The cream of claim 36, wherein topical administration of about 2 grams to about 4 grams of the cream comprising 1 wt. % of KM-001 to about 400 cm² to about 600 cm² of the skin of a patient in need thereof provides about 100 μg/g to about 3500 μg/g of the KM-001 in hypodermis, as measured by MALDI.

43. The cream of claim 36, wherein topical administration of about 2 grams to about 4 grams of the cream comprising 1 wt. % of KM-001 to about 400 cm² to about 600 cm² of the skin of a patient in need thereof provides about 50 μg/g to about 1200 μg/g of the KM-001 in hair follicles, as measured by MALDI.

44. The cream of any one of the preceding claims, wherein topical administration to the skin of a patient in need thereof provides penetration of about 0.05 wt. % to about 20 wt. % of the KM-001 presented in the applied composition in the skin into stratum corneum, epidermis, or dermis, as measured by in vitro penetration test (IVPT).

45. The cream of claim 44, wherein the topical administration to the skin of a patient in need thereof provides penetration of about 1 wt. % to about 10 wt. % of the KM-001 presented in the applied composition in the skin into the stratum corneum, as measured by IVPT.

46. The cream of claim 44, wherein the topical administration to the skin of a patient in need thereof provides penetration of about 0.2 wt. % to about 8 wt. % of the KM-001 presented in the applied composition in the skin into the epidermis, as measured by IVPT.

47. The cream of claim 44, wherein the topical administration to the skin of a patient in need thereof provides penetration of about 0.05 wt. % to about 3 wt. % of the KM-001 presented in the applied composition in the skin into the dermis, as measured by IVPT.

48. The cream of any one of the preceding claims, wherein the topical administration provides less than about 40 ng/mL of the maximum plasma concentration (Cₘₐₓ) of KM-001.

49. The cream of any one of the preceding claims, wherein the topical administration provides less than about 500 ng/mL*h of the area under the plasma drug concentration-tine curve (AUC) of KM-001.

50. A method of treating a skin disorder or pruritus, comprising topically administering a therapeutically effective amount of the composition of any one of the preceding claims to the skin of a patient in need thereof.
